(19) United States
(12) Patent Application Publication
(10) Pub. No.: US 2023/0201484 A1
(43) Pub. Date: Jun. 29, 2023

(54) DEVICES, SYSTEMS, AND METHODS FOR DELIVERING FLUID TO THE INNER EAR

(71) Applicant: Akouos, Inc., Boston, MA (US)

(72) Inventors: Michael McKenna, Boston, MA (US); John Connelly, Franklin, MA (US)

(21) Appl. No.: 17/923,333

(22) PCT Filed: May 26, 2021
(86) PCT No.: PCT/US2021/034352

§ 371 Date: Nov. 4, 2022

Related U.S. Application Data
(60) Provisional application No. 63/151,610, filed on Feb. 19, 2021.
(60) Provisional application No. 63/126,270, filed on Dec. 16, 2020.
(60) Provisional application No. 63/030,519, filed on May 27, 2020.

(51) Int. Cl. A61M 5/32

(52) U.S. Cl. CPC A61M 5/3286; A61M 2005/1588

(57) ABSTRACT

A device (10) to deliver fluid to an ear includes: a handle portion (12) including a proximal end and a distal end; a needle sub-assembly (26) coupled to the distal end of the handle portion (12) and including a bent needle (38); and tubing (36) coupled to the proximal end of the handle portion (12). The bent needle (38) extends through the handle portion (12) and fluidly connects directly to the tubing (36).

### CROSS REFERENCE TO RELATED APPLICATIONS

This application claims priority to and the benefit of U.S. Provisional Pat. Application Serial Nos. 63/030,519, filed May 27, 2020; 63/126,270, filed Dec. 16, 2020; and 63/151,610, filed Feb. 19, 2021, all entitled “DEVICES, SYSTEMS, AND METHODS FOR DELIVERING FLUID TO THE INNER EAR,” the disclosures of which are incorporated herein by reference in their entirety.

### BACKGROUND

Delivery of therapeutic agents to the inner ear presents significant challenges. The relevant organs are buried deep within the skull, encased in bone, and isolated from the blood circulatory system by a blood-cochlear barrier. Some of the organs of the inner ear including the organ of Corti are particularly inaccessible and fragile.

Fluids containing therapeutic agents can be delivered to the middle ear cavity with the hope that they diffuse across the round window membrane (RWM) into the inner ear. However, only a very small percentage of the administered fluid and therapeutic agent actually enters the fluid space of the inner ear. Distribution throughout the inner ear generally relies on simple diffusion, which causes the delivered fluid and therapeutic agent to be highly diluted by the time it reaches a target site of action in the inner ear.

### SUMMARY

The present disclosed embodiments include devices, systems, and methods for delivery of fluids into the inner ear. The devices, systems, and methods described herein include a device that administers fluid to the perilymph fluid of the inner ear. In some embodiments, the described devices, systems, and methods afford potential advantages over available devices, systems and methods, both with respect to safety and efficacy of a fluid including a therapeutic agent administered via the intracochlear route.

In some embodiments, design elements of the described devices and systems include maintenance of sterility of injected fluid; minimization of bubbles introduced to the inner ear; ability to deliver small volumes precisely at a controlled flow rate (for example, when coupled with the use of a standard pump); allowance for visualization of the round window membrane (RWM) during delivery through the external auditory canal by a surgeon; minimization of damage to the RWM, or to inner ear structures beyond the RWM; and minimization of test article leaking through the RWM.

In one aspect, the present disclosure provides a device to deliver fluid to an ear including: a handle portion including a proximal end and a distal end; a needle sub-assembly coupled to the distal end of the handle portion and including a bent needle; and tubing coupled to the proximal end of the handle portion. The bent needle extends through the handle portion and fluidly connects directly to the tubing.

In some embodiments, the device includes a telescoping support coupled to a proximal end of the needle sub-assembly.

In some embodiments, the distal end of the handle is coupled to a proximal end of the telescoping support.

In some embodiments, the telescoping support includes multiple nested hypotubes.

In some embodiments, the bent needle includes: an angled tip for piercing at least one membrane; and a bent portion.

In some embodiments, the device includes a strain relief feature coupled to the proximal end of the handle portion.

In some embodiments, the device includes a camera (for example, a distal tip camera). The distal tip camera is positioned within the needle sub-assembly.

In some embodiments, the tubing couples to the bent needle within a hollow interior of the handle portion.

In some embodiments, the device includes an inner diameter from about 0.005 inches to about 0.01 inches.

In some embodiments, the bent portion has a length from about 0.5 mm to about 5 mm (for example, from about 1 mm to about 3 mm, e.g., about 1.4 mm).

In some embodiments, the angle is from about 20 degree to about 70 degrees (e.g., from about 20 degrees to about 60 degrees, e.g., from about 20 degrees to about 50 degrees, e.g., from about 20 degrees to about 40 degrees, e.g., about 30 degrees, e.g., from about 30 degrees to about 70 degrees, e.g., from about 40 degrees to about 60 degrees, e.g., about 55 degrees).

In some embodiments, the angle is about 30 degrees.

In some embodiments, the angle is about 55 degrees.

In some embodiments, the bent needle includes a gauge within a range from about 10 to about 35, e.g., from about 20 to about 35, e.g., from about 30 to about 35, e.g., a 33 gauge.

In some embodiments, the bent needle is at least partially composed of stainless steel.

In some embodiments, the device includes an adhesive disposed on the proximal end and distal end of the handle portion.

In some embodiments, the device includes a stopper coupled to the bent needle. The stopper is shaped and sized for positioning within the inner ear and controlling a distance that the angled tip projects into a cochlea.

In some embodiments, the stopper includes a cylinder-disk shape.

In some embodiments, the stopper is molded in place onto the bent needle, and the stopper prevents the bent needle from being inserted into at least one membrane beyond a desired amount.

In some embodiments, the stopper is positioned at a distance of about 0.2 mm to about 1.2 mm (e.g., from about 0.4 mm to about 1.0 mm, e.g., from about 0.6 mm to about 0.9 mm, e.g., about 0.85 mm) from a distal end of the angled tip.

In some embodiments, the stopper includes a diameter from about 0.2 mm to about 1.2 mm (e.g., from about 0.4 mm to about 1.0 mm, e.g., from about 0.6 mm to about 0.9 mm, e.g., about 0.85 mm).

In some embodiments, the stopper includes a height from about 0.2 mm to about 1.0 mm (e.g., from about 0.3 mm to about 0.7 mm, e.g., from about 0.4 mm to about 0.6 mm, e.g., about 0.5 mm).

In some embodiments, each hyptotube of the multiple nested hypotubes includes a gauge from about 10 to about 30. (e.g., 14XH, 20TW, 23XTW, and/or 27TW).

In some embodiments, each hyptotube of the multiple nested hypotubes includes stainless steel.

In some embodiments, the handle portion further includes a tapered portion disposed at the distal end of the handle, the telescoping support coupling to the tapered portion. The handle tapers down to the first distal end (e.g., such that the second proximal end of the telescoping support is coupled to the first distal end).

In some embodiments, the telescoping support tapers from an outer diameter of about 0.2 inches or less at a proximal end to an outer diameter of about 0.01 inches or more at a distal end.

In some embodiments, the handle portion includes machined grooves for tactility and control.

In some embodiments, the handle portion is shaped and sized to facilitate placement into the inner ear.

In some embodiments, the strain relief feature includes layered extrusions (e.g., layered Pebax extrusions).

In some embodiments, the strain relief feature prevents kinking and/or deformation of the tubing.

In some embodiments, the tubing is coupled to the bent needle via compression fit.

In some embodiments, the tubing includes polyether ether ketone (PEEK).

In some embodiments, the tubing includes an inner diameter from about 0.003 inches to about 0.01 inches (e.g., about 0.007 inches).

In some embodiments, the tubing includes an outer diameter from about 1/64 inches to about 1/16 inches (e.g., about 1/32 inches).

In some embodiments, the tubing includes a length greater than 20 inches, e.g., greater than 30 inches, e.g., greater than 40 inches, e.g., greater than 50 inches, e.g., greater than 60 inches, e.g., about 60 inches.

In some embodiments, the device is sterile and/or biocompatible.

In some embodiments, the angled tip projects from the bent portion of the bent needle to form an outlet for dispensing fluid.

In another aspect, the present disclosure provides a system including the device and a sterilized syringe fluidly coupled to the tubing.

In some embodiments, the system includes a pump.

In some embodiments, the pump controls a flow rate of a fluid through any one of the devices (e.g., at a rate from about 10 µL/min to about 60 µL/min, e.g., from about 15 µL/min to about 55 µL/min, e.g., from about 20 µL/min to about 50 µL/min, e.g., from about 25 µL/min to about 45 µL/min, e.g., from about 25 µL/min to about 40 µL/min, e.g., from about 20 µL/min to about 35 µL/min, e.g., about 30 µL/min) (e.g., at a rate from about 10 µL/min to about 200 µL/min, e.g., from about 20 µL/min to about 180 µL/min, e.g., from about 30 µL/min to about 180 µL/min, e.g., from about 40 µL/min to about 150 µL/min, e.g., from about 50 µL/min to about 150 µL/min, e.g., from about 60 µL/min to about 140 µL/min, e.g., from about 70 µL/min to about 130 µL/min, e.g., from about 80 µL/min to about 120 µL/min, e.g., from about 90 µL/min to about 110 µL/min, e.g., about 100 µL/min).

In some embodiments, the stopper is seated around a stopper anchoring groove disposed within the bent needle.

In some embodiments, the device includes an annular brace disposed at the interface between the telescoping support and the handle portion.

In some embodiments, the device includes at least one machined barb disposed at the proximal end of the handle portion.

In some embodiments, the machined barb interfaces with the strain relief feature and prevents axial movement between the handle portion and the strain relief feature.

In another aspect, the present disclosure provides a delivery system including: a delivery device including: a distal end; and a stopper disposed at the distal end of the delivery device; a distal tip camera disposed at the distal end of the delivery device, the distal tip camera including an image sensor; and a monitor operatively coupled to the distal tip camera. The monitor displays information received from the distal tip camera.

In some embodiments, the stopper is transparent.

In some embodiments, the stopper includes a transparent portion for the distal tip camera to see through the stopper.

In some embodiments, the distal tip camera is disposed above the front surface of the stopper, and the front surface of the stopper faces toward a target.

In some embodiments, the target is a part of an ear.

In some embodiments, the distal tip camera is embedded (e.g. integrated) within the stopper.

In some embodiments, the distal tip camera is disposed behind the stopper.

In some embodiments, the delivery system includes a wire that is operatively coupled between the distal tip camera and the monitor.

In some embodiments, the distal tip camera includes autofocus features.

In some embodiments, the distal tip camera includes at least one of a cuboid shape, a chip shape, a cylindrical shape, and combinations thereof.

In some embodiments, the image sensor includes a field of view from about 90° to about 150°.

In some embodiments, the image sensor includes a cuboid shape with dimensions of up to 10 mm × 10 mm with a height of up to 100 mm, and/or a cylindrical shape including an outer diameter of up to 10 mm with a length of up to 100 mm.

In some embodiments, the image sensor includes an image array capable of capturing at least 10 × 10 pixels resolution video at a frame rate of at least 5 frames per second (fps).

In some embodiments, the image sensor includes an image area of at most 10 mm × 10 mm.

In some embodiments, the image sensor includes an optical format of up to 10 mm, and a pixel size of up to 10 µm.

In some embodiments, the delivery system includes a processor operatively coupled to the image sensor.

In some embodiments, the delivery system includes driver packages and/or software packages.

In some embodiments, the delivery system includes at least one light source.

In some embodiments, the delivery system includes an optical fiber.

In another aspect, the present disclosure provides a distal tip camera system including: an image sensor disposed at a distal end of a needle including a stopper; a wire operatively coupled to the image sensor; a processor operatively coupled to the image sensor; and a monitor operatively coupled to the processor to display information captured by the image sensor and processed by the processor.

In another aspect, the present disclosure provides a surgical procedure for delivering a therapeutic fluid to a portion of the inner ear (for example, using any one of the devices disclosed herein) including: developing a posterior tympanomeatal flap; creating an opening in the stapes footplate; piercing the round window with a needle positioned at the distal end of a fluid delivery device; positioning the fluid delivery device at a desired insertion depth within the round window; and flowing the therapeutic fluid through the fluid delivery device to the inner ear.

In some embodiments, the procedure includes: activating a distal tip camera, an endoscope, and/or an operating microscope prior to piercing the round window; and monitoring a flow rate of therapeutic fluid and/or a distribution of therapeutic fluid across the inner ear via the distal tip camera, the endoscope, and/or the operating microscope prior to piercing the round window.

In some embodiments, the procedure includes activating a distal tip camera prior to piercing the round window. The distal tip camera is communicatively coupled to at least one monitor viewable by a surgeon during the procedure.

In some embodiments, developing a posterior tympanomeatal flap includes cutting the posterior tympanomeatal using a micro curette and/or a drill.

In some embodiments, the procedure includes prepping and draping the ear prior to developing the posterior tympanomeatal flap; positioning the patient prior to prepping and draping the ear; inducing anesthesia prior to positioning the patient; and marking the ear prior to inducing anesthesia.

In some embodiments, the procedure includes connecting tubing between the fluid delivery device and an upstream pump prior to developing the posterior tympanomeatal flap; sterilizing the fluid delivery device prior to developing the posterior tympanomeatal flap; and priming the system prior to developing the posterior tympanomeatal flap.

In some embodiments, the therapeutic fluid includes at least one viral gene therapy.

In some embodiments, the procedure includes removing the fluid delivery device from the inner ear after flowing the therapeutic fluid through the fluid delivery device; and applying at least one skin treatment to the round window membrane and/or the stapes footplate after removing the fluid delivery device.

In some embodiments, the procedure includes returning the posterior tympanomeatal flap back to the original position after applying at least one skin treatment.

In some embodiments, the procedure includes removing bone from the junction of the bony canal and the tympanic membrane, and/or pseudomembrane overhanging bone, after developing the posterior tympanomeatal flap.

In some embodiments, the procedure includes using a diamond drill and/or an otologic drill to remove bone.

In some embodiments, creating an opening in the stapes footplate includes creating an opening in the stapes footplate using a laser.

In some embodiments, the laser includes an otologic laser.

In some embodiments, the procedure includes applying at least one of an anesthetic and an adrenaline to the ear canal of the patient prior to developing the posterior tympanomeatal flap.

In some embodiments, prepping the ear further includes applying at least one antiseptic to the ear.

In some embodiments, the antiseptic includes povidone-iodine, iodopovidone, betadine, wokadine, and/or pyodine.

In some embodiments, the skin treatment includes sodium hyaluronate and/or hyaluronic acid.

In another aspect, the present disclosure provides a method for delivering a therapeutic fluid to a portion of the inner ear (for example, using any one of the devices or systems disclosed herein) including: creating an opening in the stapes footplate; piercing the round window with a needle positioned at the distal end of a fluid delivery device; positioning the fluid delivery device at a desired insertion depth within the round window; and flowing the therapeutic fluid through the fluid delivery device to the inner ear. The therapeutic fluid includes at least one viral gene therapy.

In another aspect, the present disclosure provides a method for delivering a therapeutic fluid to a portion of the inner ear (for example, using any one of the devices or systems disclosed herein including: creating an opening in the stapes footplate; piercing the round window with a needle positioned at the distal end of a fluid delivery device; positioning the fluid delivery device at a desired insertion depth within the round window; and flowing the therapeutic fluid through the fluid delivery device to the inner ear. The desired insertion depth includes a depth from about 0.7 mm to about 1.0 mm.

In some embodiments, flowing the therapeutic fluid through the fluid delivery device to the inner ear includes flowing the therapeutic fluid at a flow rate from about 20 µL/min to about 100 µL/min.

In some embodiments, flowing the therapeutic fluid through the fluid delivery device to the inner ear includes flowing a total volume of therapeutic fluid in a range from about 0.07 mL to about 0.11 mL.

In some embodiments, flowing the therapeutic fluid through the fluid delivery device to the inner ear includes flowing therapeutic fluid for a time duration in a range from about 1 minute to about 5 minutes.

In another aspect, the present disclosure provides a device to deliver fluid to an ear including: a handle portion including a proximal end and a distal end; a telescoping support coupled to the distal end of the handle portion; a needle sub-assembly coupled to the distal end of the telescoping support, the needle sub-assembly including a bent needle; and tubing coupled to the proximal end of the handle portion.

In another aspect, the present disclosure provides a packaging system for holding a delivery device that includes a distal end and a stopper disposed at the distal end of the delivery device. The packaging system includes: a mounting surface; and a device nesting for holding the delivery device. The device nesting is mounted on the mounting surface.

In another aspect, the present disclosure provides a packaging system for holding a delivery device, the packaging system including: a mounting surface; and a device nesting for holding the delivery device. The device nesting is mounted on the mounting surface.

In some embodiments, the system includes at least one pair of oppositely-oriented slits disposed within the mounting surface.

In some embodiments, the pair of oppositely-oriented slits holds tubing fluidly coupled to a proximal end of the delivery device.

In some embodiments, the system includes a plurality of nesting notches disposed within the device nesting, the nesting notches holding at least one of a proximal end, a distal end, and a body portion of the delivery device.

In some embodiments, the system includes at least one attachment slit disposed within the mounting surface; and at least one locking portion extending across the device nesting and attaching to the attachment slit.

In some embodiments, the device nesting is fiddle-shaped.

In some embodiments, the system includes at least one twist-tie for securing the delivery device to the device nesting.

In some embodiments, the pair of oppositely-oriented slits includes a pair of curved ends at either end to prevent the pair of oppositely-oriented slits from causing damage to the mounting surface.

In some embodiments, the delivery device includes: a device body including a distal tip and a proximal end; and tubing fluidly coupled to the proximal end.

In another aspect, the present disclosure provides a packaging system for holding a delivery device used to deliver therapeutic fluid to the inner ear, the packaging system including: a mounting surface; and a device nesting for holding the delivery device. The device nesting is mounted to the mounting surface.

In some embodiments, the system includes PEEK tubing fluidly coupled upstream of the delivery device; and a sleeve disposed around the PEEK tubing.

In some embodiments, the system includes a sleeve disposed concentrically around the tubing to prevent kinking of the tubing.

In some embodiments, the sleeve is composed of a polymer material.

In another aspect, the present disclosure provides a surgical procedure for delivering a therapeutic fluid to a portion of the inner ear of a patient including: injecting the therapeutic fluid via a delivery device as described herein into the inner ear.

In some embodiments, a surgical procedure includes performing a transcanal tympanotomy; performing a laser-assisted micro-stapedotomy; and injecting the therapeutic fluid via a delivery device as described herein into the inner ear.

In some embodiments, a surgical procedure includes performing a transcanal tympanotomy; performing a laser-assisted micro-stapedotomy; injecting the therapeutic fluid via a delivery device as described herein into the inner ear; applying sealant around the round window and/or an oval window of the patient; and lowering a tympanomeatal flap of the patient to the anatomical position.

In some embodiments, a surgical procedure includes performing a transcanal tympanotomy; preparing a round window of the patient; performing a laser-assisted micro-stapedotomy; preparing both a delivery device as described herein and the therapeutic fluid for delivery to the inner ear; injecting the therapeutic fluid via the delivery device into the inner ear; applying sealant around the round window and/or an oval window of the patient; and lowering a tympanomeatal flap of the patient to the anatomical position.

In some embodiments, performing a laser-assisted micro-stapedotomy includes using a KTP otologic laser and/or a CO₂ otologic laser.

In some embodiments, the therapeutic fluid includes an AAV vector. In some embodiments, the AAV vector is an Anc80 AAV vector. In some embodiments, the AAV vector comprises a coding region encoding hOTOF.

Throughout the description, where devices, systems, procedures, and/or methods are described as having, including, or comprising specific components, or where methods are described as having, including, or comprising specific steps, it is contemplated that, additionally, there are devices, systems, procedures, and/or methods of the present disclosure that consist essentially of, or consist of, the recited components, and that there are methods according to the present disclosure that consist essentially of, or consist of, the recited processing steps.

It should be understood that the order of steps or order for performing certain actions is immaterial as long as the method remains operable. Moreover, two or more steps or actions may be conducted simultaneously.

The following description is for illustration and exemplification of the disclosure only, and is not intended to limit the disclosure to the specific embodiments described.

The mention herein of any publication, for example, in the Background section, is not an admission that the publication serves as prior art with respect to any of the present claims. The Background section is presented for purposes of clarity and is not meant as a description of prior art with respect to any claim.

### BRIEF DESCRIPTION OF THE DRAWING

A full and enabling disclosure of the present disclosed embodiments, including the best mode thereof, directed to one of ordinary skill in the art, is set forth in the specification, which makes reference to the appended figures, in which:

FIG. 1 illustrates a perspective of a device for delivering fluid to an inner ear, according to aspects of the present disclosed embodiments;

FIG. 2 illustrates a sideview of a bent needle sub-assembly, according to aspects of the present disclosed embodiments;

FIG. 3 illustrates a perspective view of a device for delivering fluid to an inner ear, according to aspects of the present disclosed embodiments;

FIG. 4 illustrates a perspective view of a bent needle sub-assembly coupled to the distal end of a device, according to aspects of the present disclosed embodiments;

FIG. 5 depicts a device coupled to tubing, according to aspects of the present disclosed embodiments;

FIG. 6 depicts a device coupled to a strain release feature, according to aspects of the present disclosed embodiments;

FIG. 7 illustrates a perspective view of a telescoping hypotube needle support, a needle, and a stopper, according to aspects of the present disclosed embodiments;

FIG. 8 illustrates a sideview of a needle, according to aspects of the present disclosed embodiments;

FIG. 9 illustrates a perspective of a telescoping hypotube needle support, according to aspects of the present disclosed embodiments;

FIG. 10 illustrates a perspective of a strain release feature, according to aspects of the present disclosed embodiments;

FIG. 11 illustrates a perspective of tubing (or hooping), according to aspects of the present disclosed embodiments;

FIG. 12 illustrates a device for delivering fluid to an inner ear, according to aspects of the present disclosed embodiments;

FIG. 13 illustrates a perspective of the bent needle sub-assembly, according to aspects of the present disclosed embodiments;

FIG. 13A illustrates a perspective of the bent needle sub-assembly, according to aspects of the present embodiments;

FIG. 13B illustrates a perspective of the bent needle sub-assembly, according to aspects of the present embodiments;

FIG. 13C illustrates a side view of a device for delivering fluid to an inner ear, according to aspects of the present disclosed embodiments;

FIG. 13D illustrates a side view of a device for delivering fluid to an inner ear, according to aspects of the present disclosed embodiments;

FIG. 13E illustrates a side view of a device for delivering fluid to an inner ear, according to aspects of the present disclosed embodiments;

FIG. 13F illustrates a side view of a device for delivering fluid to an inner ear, according to aspects of the present disclosed embodiments;

FIG. 13G illustrates a side view of a device for delivering fluid to an inner ear, according to aspects of the present disclosed embodiments;

FIG. 13H illustrates a side view of a device for delivering fluid to an inner ear, according to aspects of the present disclosed embodiments;

FIG. 14 depicts a packaged device in casing coupled to tubing, according to aspects of the present disclosed embodiments;

FIG. 14A depicts a packaged device in alternate packaging coupled to tubing, according to aspects of the present embodiments;

FIG. 15 illustrates a perspective view of a distal tip camera disposed within a system, according to aspects of the present embodiments;

FIG. 16 illustrates a perspective view of a distal tip camera disposed within a system, according to aspects of the present embodiments;

FIG. 17 illustrates a perspective view of a distal tip camera disposed within a system, according to aspects of the present embodiments;

FIG. 18 illustrates a perspective view of a distal tip camera disposed within a system, according to aspects of the present embodiments;

FIG. 19 illustrates a side view of a distal tip camera, according to aspects of the present embodiments;

FIG. 20 illustrates a side view of a distal tip camera, according to aspects of the present embodiments;

FIG. 21 illustrates a side view of a distal tip camera, according to aspects of the present embodiments;

FIG. 22 illustrates a side view of a distal tip camera, according to aspects of the present embodiments;

FIG. 23 illustrates a side view of an optical fiber, according to aspects of the present embodiments;

FIG. 23A illustrates an embodiment of a device, according to aspects of the present embodiments;

FIG. 23B illustrates an embodiment of a device, according to aspects of the present embodiments;

FIG. 23C illustrates an embodiment of a device, according to aspects of the present embodiments;

FIG. 23D illustrates an embodiment of a device, according to aspects of the present embodiments;

FIG. 23E illustrates an embodiment of a device, according to aspects of the present embodiments;

FIG. 23F illustrates an embodiment of a device, according to aspects of the present embodiments;

FIG. 23G illustrates an embodiment of a device, according to aspects of the present embodiments;

FIG. 23H illustrates an embodiment of a device, according to aspects of the present embodiments;

FIG. 24 illustrates a side view of a delivery system, according to aspects of the present embodiments; and

FIG. 25 illustrates a method for delivering a therapeutic fluid, according to aspects of the present embodiments.

### DESCRIPTION OF CERTAIN EMBODIMENTS

Reference will now be made in detail to the present disclosed embodiments, one or more examples of which are illustrated in the accompanying drawing. The detailed description uses numerical and/or letter designations to refer to features in the drawing. Like or similar designations in the drawing and description have been used to refer to like or similar parts of the present embodiments.

The devices, systems, and methods described herein afford potential advantages over off-the-shelf materials and other delivery systems, both with respect to safety and efficacy of a therapeutic agent. For example, the described devices and systems were specifically designed for intracochlear route of administration. In some embodiments, design elements of the described device may include: maintenance of sterility of injected fluid; minimization of air bubbles introduced to the inner ear; ability to precisely deliver small volumes at a controlled rate; delivery through the external auditory canal by the surgeon; minimization of damage to the round window membrane (RWM), or to inner ear, e.g., cochlear structures beyond the RWM; and minimization of injected fluid leaking back out through the RWM.

The devices, systems, and methods provided herein also describe the potential for delivering fluids safely and efficiently into the inner ear, in order to treat conditions and disorders that would benefit from delivery of fluids to the inner ear, including, but not limited to, hearing and balance disorders or intracranial tumors such as vestibular schwannoma. As another example, by placing a vent in the stapes footplate and injecting through the RWM, therapeutic agents are dispersed throughout the cochlea with minimal dilution at the site of action. The development of the described devices allows the surgical administration procedure to be performed through the external auditory canal in humans. The described devices can be removed from the ear following infusion of an amount of fluid into the perilymph of the cochlea. In patients, the device may be advanced through the external auditory canal, either under surgical microscopic control or along with an endoscope.

### Devices

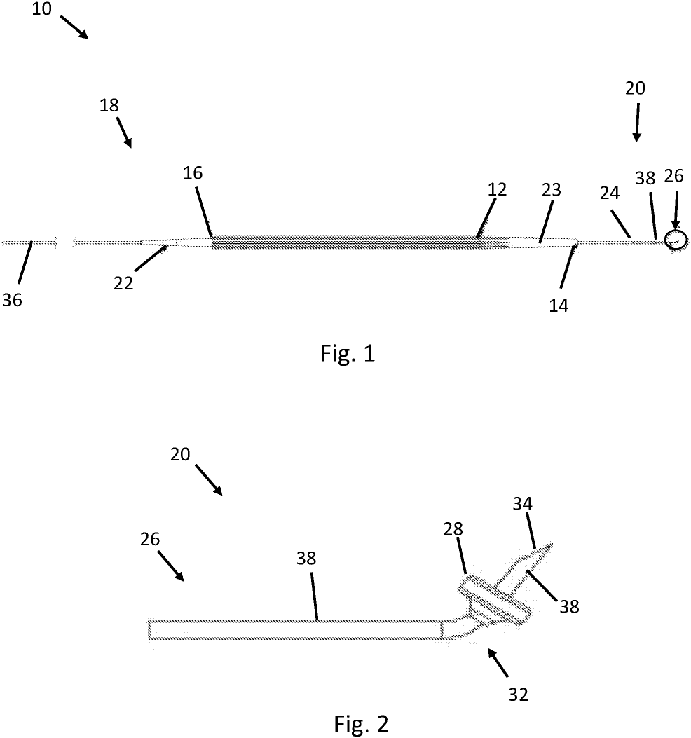

FIG. 1 illustrates an exemplary device 10 for delivering fluid to an inner ear. Device 10 includes a knurled handle 12, and a distal handle adhesive 14 (for example, an epoxy such as loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 2) with stopper 28 (shown in FIG. 2) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer.

The telescoping hypotube needle support 24 surrounds and supports a bent needle 38 (shown in FIG. 2) disposed therewithin.

Referring still to FIG. 1, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some emboidments, may be coupled to a control system and/or power supply (not shown)). In some embodiments, the bent needle 38 (shown in FIG. 2) extends from the distal end 20, through the telescoping hypotube needle support 24, threough the tapered portion 23, through the knurled handle 12, and through the strain relief feature 22 and fluidly connects directly to the tubing 36. In other embodiments, the bent needle 38 fluidly connects with the hollow interior of the knurled handle (for example, via the telescoping hypotube needle support 24) which in turn fluidly connects at a proximal end 16 with tubing 36. In embodiments where the bent needle 38 does not extend all the way through the interior of the device 10, the contact area (for example, between overlapping nested hyotubes 42), the tolerances, and/or sealants between interfacing components must be sufficent to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

FIG. 2 illustrates a sideview of the bent needle sub-assembly 26, according to aspects of the present disclosed embodiments. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 4) of the bent portion 32 may vary. A stopper 28 geometry may be cyclidrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurthethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

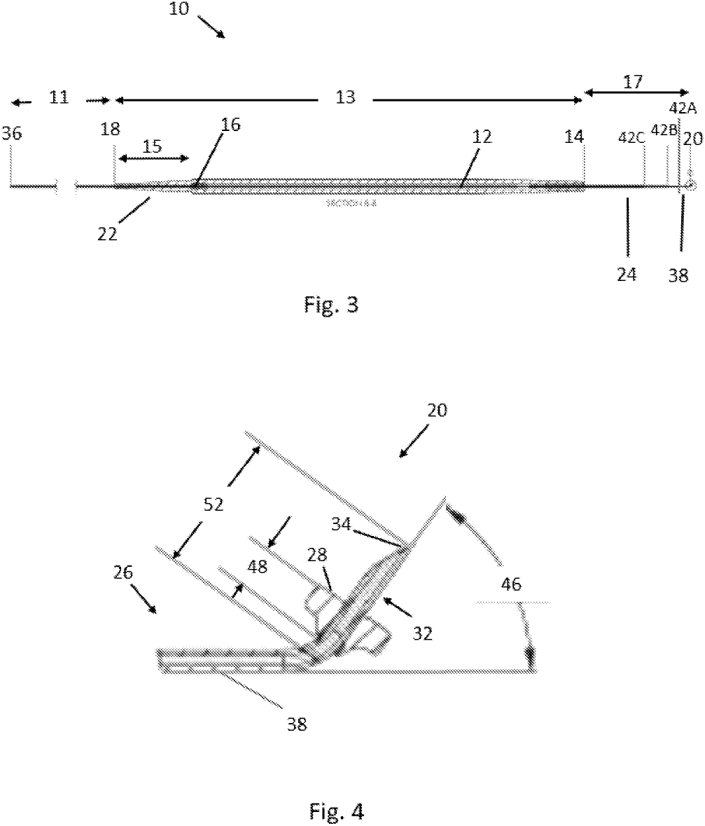

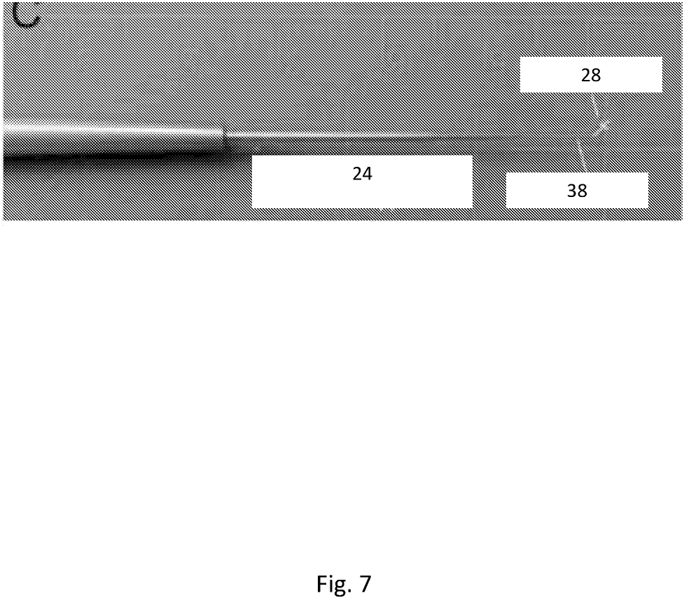

FIG. 3 illustrates a perspective view of exemplary device 10 for delivering fluid to an inner ear. Tubing 36 may be from about 1300 mm in length (dimension 11 in FIG. 3) to about 1600 mm, or from about 1400 mm to about 1500 mm, or from about 1430 mm to about 1450 mm. Strain release feature 22 may be from about 25 mm to about 30 mm in length (dimension 15 in FIG. 3), or from about 20 mm to about 35 mm in length. Handle 12 may be about 155.4 mm in length (dimension 13 in FIG. 3), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D (shown in FIG. 9). The total length of hypotubes 42A, 42B, 42C and tip assembly 26 (dimension 17 in FIG. 3) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38 (as shown in FIG. 7).

FIG. 4 illustrates a perspective view of bent needle sub-assembly 26 coupled to the distal end 20 of device 10, according to aspects of the present disclosed embodiments. As shown in FIG. 4, bent needle sub-assembly 26 may include a needle 38 coupled to a bent portion 32. In other embodiments, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 4, bent needle sub-assembly 26 may also include a stopper 28 disposed around bent portion 32. FIG. 4 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other embodiments, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 4 shows that stopper 28 may have a geometry that is cyclidrical, disk-shaped, and/or dome-shaped. A person of ordinary skill will appreciate that other geometries could be used.

The delivery of fluid to the cochlea to access the RWM in non-human primates (NHPs) differs from the approach used in human patients. For example, device 10 (as shown in FIG. 1) may be advanced through the external auditory canal in human patients, either under surgical microscopic control or along with an endoscope, an approach that is not feasible, even in larger NHPs (such as baboons).

In NHPs, an approach to access the RWM is more similar to that typically used for cochlear implant procedures in patients, which results in a slightly different angle 46 to target the RWM. For example, angle 46 as shown in FIG. 4 may be about 55 degrees for use in human patients. Alternatively, angle 46 as shown in FIG. 4 may be about 30 degrees in NHPs. In other embodiments, angle 46 may be from about 1 degree to about 70 degrees. In other embodiments, angle 46 may be from about 5 degrees to about 70 degrees. In other embodiments, angle 46 may from about 20 degrees to about 70 degrees. In other embodiments, angle 46 may be from about 20 degrees to about 60 degrees. In other embodiments, angle 46 may be from about 20 degrees to about 50 degrees. In other embodiments, angle 46 may be from about 20 degrees to about 40 degrees. In other embodiments, angle 46 may be from about 30 degrees to about 70 degrees. In other embodiments, angle 46 may be from about 40 degrees to about 60 degrees. In other embodiments, angle 46 may be about 55 degrees. In some embodiments, angle 46 is adjustable across a range of angles during use of device 10.

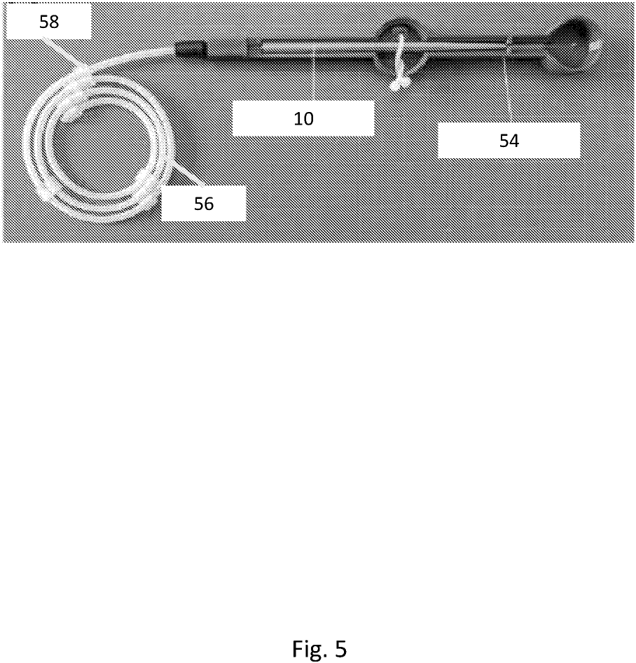

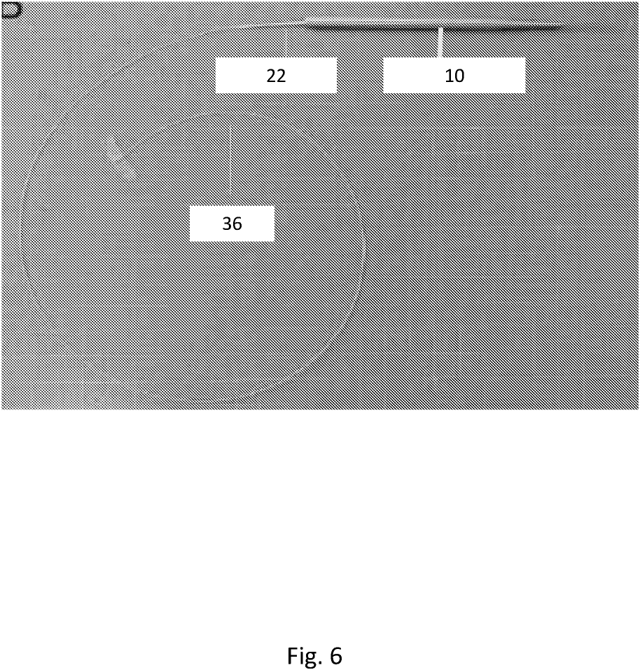

FIG. 5 depicts an exemplary device 10 with protective tube casing (or sleeve) 56 around the tubing 36 (as shown in FIG. 6) to protect it from kinking or becoming otherwise damaged. As shown in FIG. 5, device 10 may be positioned in protective device casing 54. Device casing 54 may be used to facilitate storage or handling of device 10 prior to use for fluid delivery. Tube casing 56 may include one or more cylindrical pieces 58 coupled to tube casing 56 to increase durability and reduce kinking and deformation of tube casing 56 (and hence tubing 36). The cylindrical pieces 58 may also help to keep the tube casing 56 (and hence tubing 36) in a spiral configuration during transport. In some embodiments, tube casing (or sleeve) 56 may be composed of polyether ether ketone (PEEK). In some embodiments, tube casing 56 may be composed of a thermoplastic material.

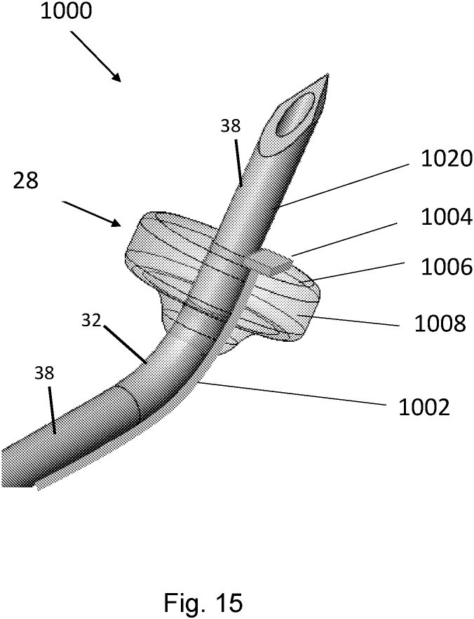

FIG. 6 depicts an exemplary device 10 coupled to strain release feature 22. Needle 38 (shown in FIG. 1) may be attached via or through telescoping hypotube needle support 24 (shown in FIG. 1) through handle 12 (shown in FIG. 1) to a fixed length of tubing 36 that may be attached to a syringe 60 (shown in FIG. 15) used to hold device 10.

FIG. 7 illustrates a perspective view of the telescoping hypotube needle support 24, needle 38, and stopper 28 of device 10, according to aspects of the present disclosed embodiments. In some embodiments, needle 38 may be concentrically disposed within the most narrow portion of the telescoping hypotube needle support 24.

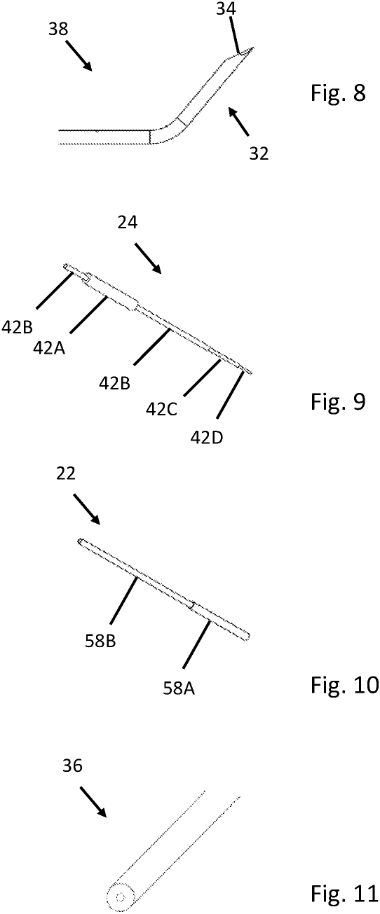

FIG. 8 illustrates a sideview of needle 38, according to aspects of the present disclosed embodiments. Needle 38 may include a bent portion 32. The bent portion 32 may include an angled tip 34 for piercing a membrane of the ear (for example, the RWM). Needle 38 may be a 33 gauge needle. Other gauges may also be used such as gauges from about 32 to about 34, or from about 31 to about 35. At finer gauges, care must be taken to ensure the needle 38 is not damaged. Needle 38 may be made out of stainless steel (for example, 304 stainless steel). Needle 38 may also be made out of any material that has similar material properties to stainless steel (such as strength or other mechanical properties). For example, needle 38 may be composed of titanium. Needle 38 may have a bent length of about 1.45 mm, or from about 1.2 mm to about 1.7 mm (as shown in FIG. 4) and an angle of about 55 degrees, or from about 40 degrees to about 70 degrees (as shown in FIG. 4), or from about 20 degrees to about 70 degrees and other sub ranges therebetween include from about 25 degrees to about 45 degrees.

FIG. 9 illustrates a perspective of a telescoping hypotube needle support 24, according to aspects of the present disclosed embodiments. In some embodiments, telescoping hypotube needle support 24 may include two or more nested hypotubes, for example four nested hypotubes 42A, 42B, 42C, and 42D (see also FIG. 3 which shows an embodiment with three nested hypotubes 42A, 42B, and 42C). Needle 38 may be the most narrow portion of the telescoping hypotube needle support 24. In other embodiments, needle 38 is disposed within the most narrow portion 42D of the telescoping hypotube needle support 24. Telescoping hypotube needle support 24 may be made out of stainless steel (for example, 304 stainless steel). Telescoping hypotube needle support 24 may also be made out of any material that has similar material properties to stainless steel (such as strength or other mechanical properties). For example, telescoping hypotube needle support 24 may be composed of titanium. Nested hypotubes 42A, 42B, 42C, 42D may include gauges of 14XH, 20TW, 23TW, and 27TW, respectively. As such, nested hypotubes 42A, 42B, 42C, 42D may include outer diameters of 0.083 inches, 0.0355 inches, 0.025 inches, and 0.014 inches, respectively, and inner diameters of 0.039 inches, 0.0255 inches, 0.017 inches, and 0.009 inches, respectively. In other embodiments, nested hypotubes 42A, 42B, 42C, 42D may include outer diameters ranging from about 0.2 inches to about 0.01 inches and inner diameters ranging from 0.08 inches to 0.004 inches. Similarly, nested hypotubes 42A, 42B, 42C, 42D may include wall thicknesses ranging from about 0.022 inches to about 0.003 inches, or from about 0.05 inches to about 0.001 inches.

Referring still to FIG. 9, needle 38 may include a gauge of from about 32 to about 34, or from about 31, to about 35, with corresponding outer diameters from about 0.01 inches to about 0.005 inches, thereby allowing it to fit with the inner-most nested hypotube 42C and/or 42D depending on the number of hypotubes. Each of the nested hypotubes 42A, 42B, 42C, 42D may also include include smoothed edges between itself and one or more of the adjacent hyoptubes, e.g., to reduce the likelihood of catching on certain anatomical components within the external auditory canal in human patients. Each of the nested hypotubes 42A, 42B, 42C, 42D may also include include an outwardly radially extending lip at a proximal end and an inwardly radially extending lip at a distal end, thereby creating interference with a neighboring nested hypotube and preventing any of the nested hypotubes 42A, 42B, 42C, 42D from becoming detached from the device 10. In other embodiments, instead of being telescopic, needle support 24 may include a single, monolithic conical member with a gradually tapering radius (for example, tapering from the radius of the outermost hypotube 42A to the radius of the innermost hypotube 42D) in place of the plurality of nested hypotubes 42A, 42B, 42C, and 42D. The telescoping hypotube needle support 24 may include nested hypotubes 42B, 42C, and 42D that extend fully through each next wider hypotube (as illustrated in FIG. 9 by hypotube 42B extending through to a distal end of hypotube 42A).

FIG. 10 illustrates a perspective view of a strain release feature 22, according to aspects of the present disclosed embodiments. Strain release feature 22 may include layered extrusions 58A and 58B. Layered extrusions 58A and 58B may include layered Pebax extrusions. Layered extrusions 58A and 58B may prevent kinking and/or deformation of PEEK tubing 36 (as shown in FIGS. 5-6) at a proximal end 18 of the knurled handle 12 (as shown in FIG. 1).

FIG. 11 illustrates a perspective view of tubing 36, according to aspects of the present disclosed embodiments. Tubing 36 may be made out of PEEK. Tubing 36 may also be composed of other materials such as thermoplastics. Tubing 36 may have an inner diameter of about 0.007 inches, or from about 0.005 inches to 0.01 inches Tubing 36 may have an outer diameter of about 1/32 inches or from about 0.02 inches to about 0.05 inches. Tubing 36 may have a length of about 60 inches, or from about 30 inches to about 100 inches. In embodiments of the device 10 in which the bent needle 38 extends all the way through the knurled handle 12 and fluidly connects directly to the tubing 36, the proximal end of the bent needle 38 may be coupled to the tubing 36 (for example, with the bent needle 38 being inserted into the tubing 36) via compression fit, adhesive, ring clamp, and other suitable connections.

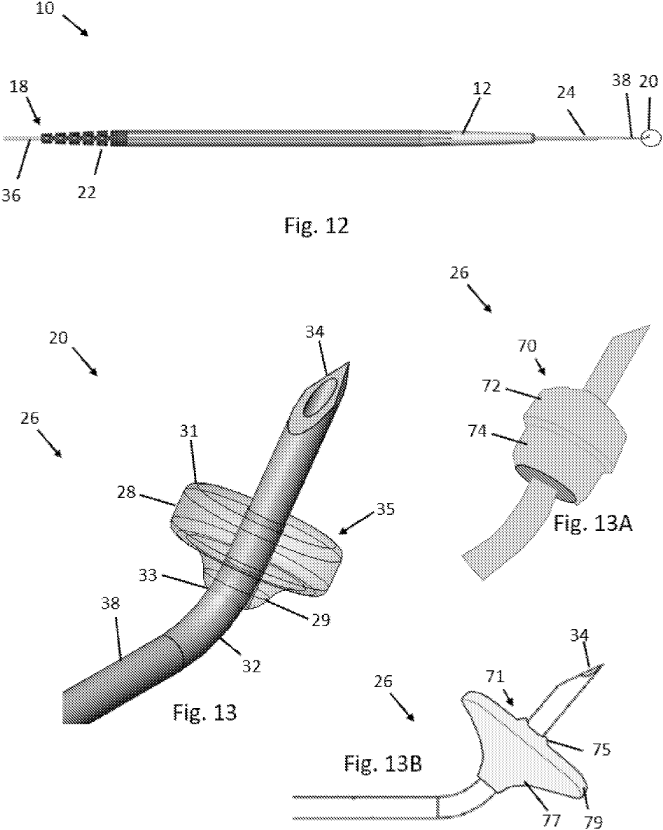

FIG. 12 illustrates an exemplary device 10 for delivering fluid to an inner ear including the telescoping hypotube needle support 24, the bent needle 38 at the distal end 20 of the device 10. As shown in FIG. 12, device 10 may include an alternate embodiment of the strain release feature 22 that may add flexibility to the interface between the tubing 36 and the proximal end 18 of the device. The embodiment of the strain release feature 22 of FIG. 12 may also reduce kinking or deformation of tubing 36. Strain realease feature 22 may also provide durability where tube casing 56 (as shown in FIG. 5) interfaces with the proximal end 18 of handle 12.

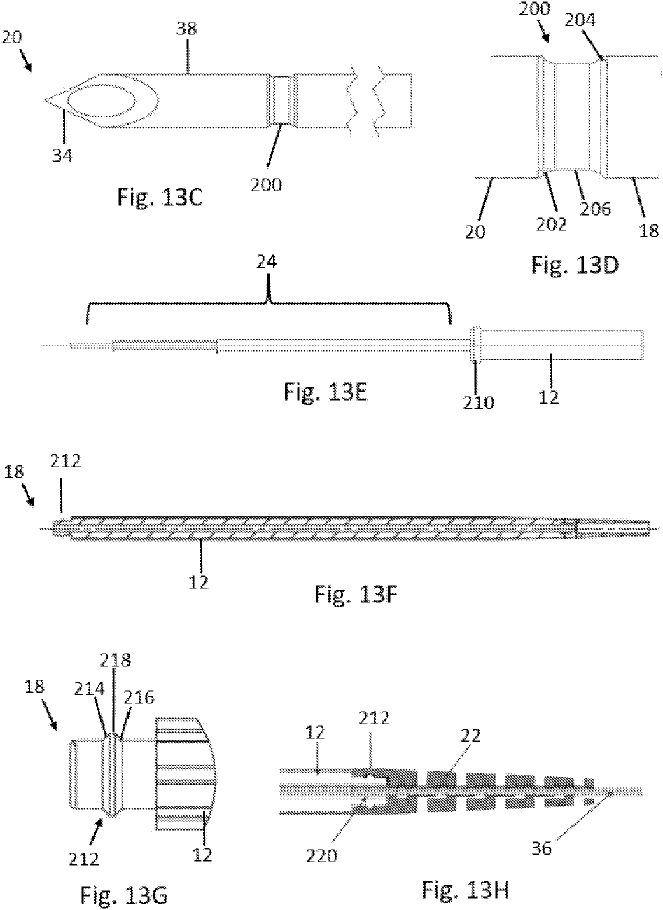

FIG. 13 illustrates a perspective of the bent needle sub-assembly 26, according to aspects of the present disclosed embodiments. The needle sub-assembly 26, located at the distal end 20 of the device 10, may include an angled tip 34. The stopper 28 may be disposed around the needle 38 such that a stopper proximal end 33 is adjacent the bent portion 32 of the needle 38. The stopper 28 may include a stopper tapered portion 29 with a gradually increasing radius from the stopper proximal end 33 toward the stopper distal end 35. The stopper may also include one or more chamfers (for example, chamfer 31 at the stopper distal end 35).

FIG. 13A illustrates a perspective view of the bent needle sub-assembly 26 including an alternate stopper 70 design, according to aspects of the present disclosed embodiments. The alternate stopper 70 of FIG. 13A may be more rounded as compared to the stopper 28 of FIG. 13, which may be more disk-shaped or donut-shaped. For example, the alternate stopper 70 may include a maximum outer diameter that is approximately equal to its maximum length, or that is from about 0.75 to about 1.5 times its maximum length. By contrast, the stopper 28 illustrated in FIG. 13 may include a maximum diameter that is about twice the maximum length, or that is from about 1.5 to about 2.5 times the maximum length. The alternate stopper 70 may also include a flexible portion 72 that is rounded or curved (i.e., convex) towards the distal end of the bent needle sub-assembly 26. The alternate stopper 70 may also include a rigid portion 74 that is located proximate of the flexible portion 72. The rigid portion 74 may contain an outer diameter that is smaller than that of the flexible portion 72.

FIG. 13B illustrates a perspective view of the bent needle sub-assembly 26 including an alternate stopper 71 design, according to the present disclosed embodiments. The alternate stopper 71 of FIG. 13B includes a tapered portion 77 that gradually tapers from the needle to the stopper outer circumerence 79. The alternate stopper 71 may also include a lip portion 75 that extends slightly toward the distal tip of the needle 34. The alternate stopper 71 may also include a thinner aspect ratio (for example, compared to the stoppers of FIGS. 13 and 13A) such that the largest diameter of the stopper 71 (for example, as measured at the outer circumference 79) is about 10 times greater than the minimum thickness of the stopper 71. In other embodiments, the largest diameter of the stopper 71 may be from about 7 to about 12 times greater than the minimum thickness of the stopper 71, or from about 5 to about 15 times greater than the minimum thickness of the stopper 71.

FIG. 13C illustrates an embodiment of the distal end 20 of the device 10 (including the needle tip 34), according to aspects of present embodiments. In the embodiment illustrated in FIG. 13C, the device 10 includes a stopper anchoring groove 200, disposed within the device 10 at the distal end 20. The stopper anchoring groove 200 may be used to help anchor the stopper 28, 70, 71 (shown in FIGS. 2, 4, 13, 13A, 13B, and 15-18) around the needle 38 at the distal end 20.

FIG. 13D is an enlarged view of the stopper anchoring groove 200, according to aspects of the present embodiments. The stopper anchoring groove 200 may include a first declined portion 202 (for example, adjacent a distal end 20 of the stopper anchoring groove 200), a second declined portion 204 (for example, adjacent a proximal end 18 of the stop anchoring groove 200), and a recessed portion 206 (or flat portion 206) disposed axially between the first and second declined portions 202, 204 (thereby forming the stopper anchoring groove 200). The recessed portion 206 includes a smaller diameter than the rest of the needle 38. Each of the first and second declined portions 202, 204 may linearly transition from the outer circumference of the needle 38 to the recessed portion 206, and/or may be rounded (thereby forming one or more fillets).

FIG. 13E is an enlarged view of the transition between the device handle 12, and the telescoping hypotubes 24, according to aspects of the present embodiments. In the embodiment illustrated in FIG. 13E, the device may include a lip joint 210 (or brace, for example, an annular brace) that reinforces the transition between the device handle 12, and the telescoping hypotubes 24. As such, the lip joint 210 (or brace) may be disposed at an interface between the handle portion 12 and the telescoping hypotubes 24 (or support). The lip joint 210 may include a larger outer diameter than the outer diameter of the handle portion 12 such that a portion of the lip joint 210 is disposed around the handle portion 12. The lip joint 210 may also include an inner diameter that tightly fits around the outer diameter of the thickest member of the plurality of telescoping hypotubes 24.

FIG. 13F illustrates an embodiment of the device 10, according to aspects of the present embodiments. In the embodiment illustrated in FIG. 13F, the device 10 includes a machined barb 212 integrated into a proximal end 18 of the device handle 12. The machined barb 212 may be used in connection with the strain relief features 22 (shown in FIGS. 1, 3, 12, and 13H), in order to prevent the strain relief features 22 from axially detaching from the device handle 12.

FIG. 13G is an enlarged view of the machined barb 212, according to aspects of the present embodiments. The machined barb 212 may include a first inclined portion 214 (for example, on the proximal end 18 of the device handle 12), a second inclined portion 216, and a plateau portion disposed between the first and second inclined portions 214, 216.

FIG. 13H is an enlarged view of the strain relief features 22, according to aspects of the present embodiments. (The device depicted in FIG. 13H is illustrated in an opposite orientation to that of FIGS. 13F and 13G.) PEEK tubing 36 may be bonded (for example, via epoxy 220) or otherwise coupled to the device handle 12. The strain relief features 22 may be disposed around the PEEK tubing 36, which itself may be disposed around needle 38, in order to prevent kinking of (and/or other damage to) the needle 38. In addition, machined barb 212 helps to prevent the strain relief features 22 from dislodging or becoming decoupled from the device handle 212. In some embodiments, the strain relief features may be composed of molded Santoprene, among other suitable materials. In some embodiments, in order to get the PEEK tubing 36 around the needle 38, the PEEK tubing 36 may be split around the needle 38. A heat shrink sleeve (not shown) may be placed over the junction between the PEEK tubing 36 and the needle 38. The junction may then be exposed to heat. Following exposure to heat, the junction may be reflowed or reshaped such that the junction is left with a smooth final outer profile.

### Packaging System

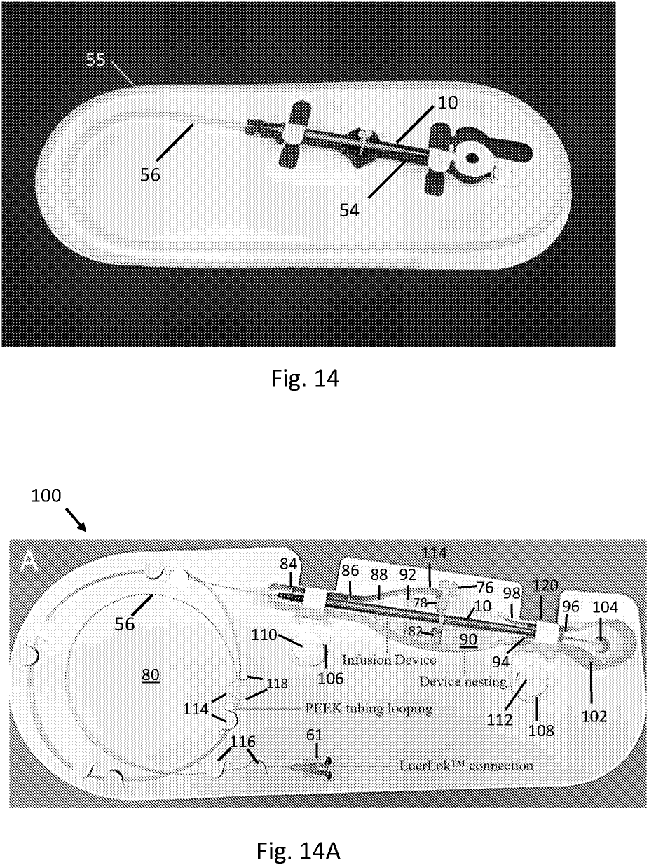

FIG. 14 depicts packaging 55 that includes device 10 encased in device casing 54 and coupled to tube casing 56. For example, packaging 55 may provide for a sterile device 10 for fluid delivery to the RWM through the external auditory canal. Device 10 may also be a single-use disposable product. In this embodiment, the device is appropriately discarded (e.g., in a biohazard_sharps container). In some embodiments, the packaging 55, tube casing 56, device casing 54, device 10, and components thereof are all constructed of materials that are robust enough to withstand gamma sterilization (for example, gamma irradiation that uses Cobalt 60 radiation to kill microorganisms and microbes). In some embodiments, the packaging 55, tube casing 56, device casing 54, device 10, and components thereof are all constructed of materials that are sufficiently resistant to temperature to withstand steam sterilization.

FIG. 14A illustrates a top perspective view of the device 10 nested in alternate packaging 100 (or packaging system 100), according to aspects of the present embodiments. The packaging (or packaging system) 100 allows for safe and sterile transport and/or shipping of the device 10. The packaging 100 may include a mounting surface 80 and device nesting 90 disposed on the mounting surface 80. The mounting surface 80 may be composed of cardboard, hardened cardstock, or polymer materials. The mounting surface 80 may also be composed of cardboard or hardened cardstock with a polymer coating. The device nesting 90 may be composed of similar materials to the mounting surface 80. The device nesting 90 may protrude out of the plane of the mounting surface 80. The mounting surface 80 may include several pairs of oppositely-oriented slits 114. The slits may be cut into the mounting surface 80 to help hold the PEEK tubing 36 in place, thereby preventing the PEEK tubing 36 (and metallic tubing therewithin) from kinking, twisting, breaking, and/or or becoming otherwise damaged. Each slit of each pair of oppositely-oriented slits 114 may include a pair of curved ends 118 at either end to prevent the slits 114 from causing rips and/or tears in the mounting surface 80 due to external forces acting on the of oppositely-oriented slits 114. The oppositely-oriented slits 114 prevent the PEEK tubing 36 (and metallic tubing therewithin) from over-kinking and/or under-kinking because the slits are disposed on either side of (i.e., radially inside of and outside of) the PEEK tubing 36 while it is coiled, thereby ensuring that a desired radius of the coiled tubing 36 is maintained. In the embodiment of FIG. 14A, the mounting surface 80 includes a total of 5 pairs of oppositely-oriented slits 114. However, in other embodiments, the mounting surface 80 may include other numbers of pairs of oppositely-oriented slits 114, as necessary, including 1, 2, 3, 4, 6, 7, 8, 9, 10 and/or more than 10.

Still referring to FIG. 14A, the alternate packaging (or packaging system) 100 may include at least one pair of oppositely-oriented slides 116 adjacent the Luer lock 61, in order to support the Luer lock 61 and prevent it from getting damaged. The device nesting 90 may be used to support the device 10 and may include several pairs of nesting notches 84, 86, 88, 92, 94, 96 that prevent the device 10 from moving laterally or longitudinally within the device nesting 90. For example, nesting notch pair 84 is positioned to hold the strain relief feature 22 (shown in FIGS. 1 and 12), nesting notch pairs 86, 88, and 92 are positioned at the proximate end of the body of device 10, and nesting notch pairs 94 and 96 are positioned toward the distal end of the device 10. The device nesting 90 may include a first contoured portion 114 near the mid-section of the body of the device 10 to accommodate a semi-flexible twist-tie 76 that is used to secure the device to the device nesting 90. The device nesting 90 may also include first and second twist-tie holes 78, 82 to allow the twist-tie 76 to extend underneath the device nesting 90, thereby extending around the bottom of a portion of the device nesting 90, in order to hold the device nesting 90 and the device 10 closely together. The device nesting 90 may also include a tip hole 104 at the distal end to protect the tip and bent needle sub-assembly of the device 10. As such, the tip of the device 10 may rest within the tip hole 104, thereby minimizing the risk of damage due to the tip making contact with any structures.

Referring still to FIG. 14A, the device nesting 90 may include a second contoured portion 102 at the distal end to accommodate the tip of the device 10 and the tip hole 104. The device nesting 90 gradually extends away from the device centerline (i.e., the center of the pairs of nesting notches 84, 86, 8, 92, 94, and 96) at each of the first and second contoured portions 114, 102 such that the device nesting 90 forms a fiddle or violin shape, including a neck portion 120 disposed longitudinally between the first and second contoured portions 114, 102. Walls of the device nesting 90 that protrude out of the plane of the mounting surface 80 may do so gradually, for example, as illustrated at taper 98 which transitions gradually from the raised protrusion (or wall) at the neck 120 down to the plane of the mounting surface 80. The packaging (or packaging system) 100 in the embodiment of FIG. 14A may also include tube casing (or sleeve) 56 (shown in FIGS. 5 and 14). The tube casing (or sleeve) 56 may be disposed concentrically around the PEEK tubing 36, and helps protect the PEEK tubing 36 and prevents it from kinking, bending, and/or becoming otherwise damaged. The tube casing (or sleeve) 56 may be placed around the PEEK tubing 36 (for example, prior to shipping or transport of the device 10 and/or system 100) and may be removed by disconnecting the Luer lock 61 from the PEEK tubing 36 and/or prior to connecting the Luer lock 61 to the PEEK tubing 36. The tube casing (or sleeve) 56 may be composed of any suitable material such as polymers such as PEEK, composite materials, metallic materials, as well as other suitable materials.

Still referring to FIG. 14A, the packaging (or packaging system) 100 may include one or more locking features including a first locking portion 106 near the proximal end of the device 10 and device nesting 90, and a second locking portion 108 near the distal end of the device 10. The second locking portion 108 may extend across the neck 120 of the device nesting 90. Each of the first and second locking portions 106, 108 extends from one side of the mounting surface 80 and across the device nesting 90 (i.e., after the device 10 been placed within the device nesting 90) to the mounting surface 80 on the other side of the device nesting 90. The first and second locking portions 106, 108 may attach to first and second attachment slits 110, 112, the first attachment slit 110 being disposed within the mounting surface 80 at the proximal end of the device nesting 90, and the second attachment slit 112 being disposed at the distal end of the device nesting 90. The first locking portion 106 interfaces with and attaches to the first attachment slit 110, while the second locking portion 108 interfaces with and attaches to the second attachment slit 112. The device nesting 90 may be coupled to the mounting surface 80 using any suitable mechanisms including epoxy, fusion, adhesion, glue, as well as other suitable means. In addition, in some embodiments, the device nesting 90 may be formed via 3D printing (for example, via fused deposition modeling (FDM), stereo-lithography (SLA), as well as other modalities). In some embodiments, the packaging 100, device nesting 90, mounting surface 80, and components thereof are all constructed of materials (for example, polymers, thermoset plastics, thermoplastics, composite materials, and other materials) that are sufficiently resistant to temperature to withstand steam sterilization, gamma irradiation (that uses Cobalt 60 radiation to kill microorganisms and microbes), and other sterilization methods.

### Systems

A system of the present disclosure may include at least one distal tip camera (DTC) for visualizing and/or monitoring the delivery of fluid to a target (for example, outer, middle, and/or inner ear). In some embodiments, the distal tip camera may be operatively coupled to the device 10, while in other embodiments, the distal tip camera may be installed as a part of the device 10 (that is, an all-in-one device). The distal tip camera may include at least one of a charge-coupled device (CCD) and a complementary metal-oxide semiconductor (CMOS). The distal tip camera may include at least one image sensor (for example, a lens) to receive, convey, and/or convert a signal (for example, an analog or a digital signal) from the target (for example, outer, middle, and/or inner ear). In some embodiments, the distal tip camera may further include at least one processor (for example, a video processor or an in-distal tip camera processor) for processing images and/or controlling the distal tip camera, while in other embodiments, the processor may be disposed separately from the distal tip camera. The distal tip camera and/or the image sensor may include a cuboid shape, a chip shape, a flattened cube shape, a cylindrical shape, and combinations thereof.

FIG. 15 illustrates a perspective view of a distal tip camera 1004 disposed above a stopper 28 within a system 1000, according to aspects of the present embodiments. The stopper 28 may be attached to an outer surface 1020 of the bent portion 32 of needle 38. The distal tip camera 1004 may be operatively coupled with a wire 1002. In some embodiments, the distal tip camera 1004 may be disposed at a front surface 1006 of the stopper 28. In some embodiments, the distal tip camera 1004 may be embeded in the front surface 1006 of the stopper 28. The front surface 1006 may face toward the target (for example, outer, middle, and/or inner ear). In some embodiments, the distal tip camera 1004 may be disposed at a side surface 1008 of the stopper 28 (not shown).

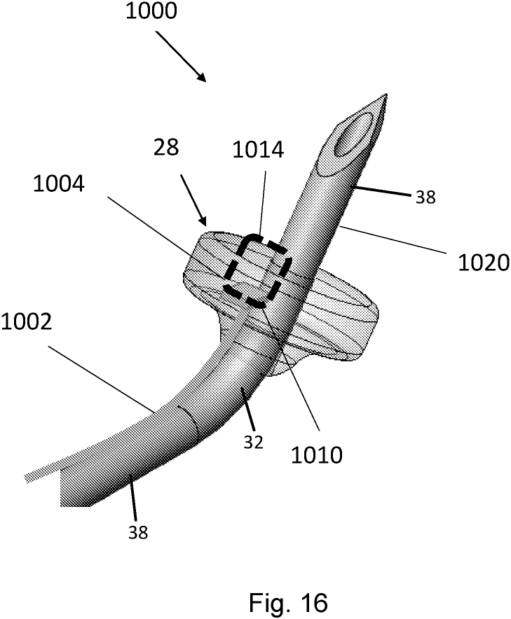

FIG. 16 illustrates a perspective view of a distal tip camera 1004 disposed in a cavity 1010 within the stopper 28 within a system 1000, according to aspects of the present embodiments. The stopper 28 may be attached to the outer surface 1020 of the bent portion 32 of needle 38. The distal tip camera 1004 may be operatively coupled with a wire 1002. The cavity 1010 may be created by cutting a portion (for example, up to 30%) of the stopper 28. In some embodiments, the cavity 1010 may be near or beside the outer surface 1020 of the device 10.

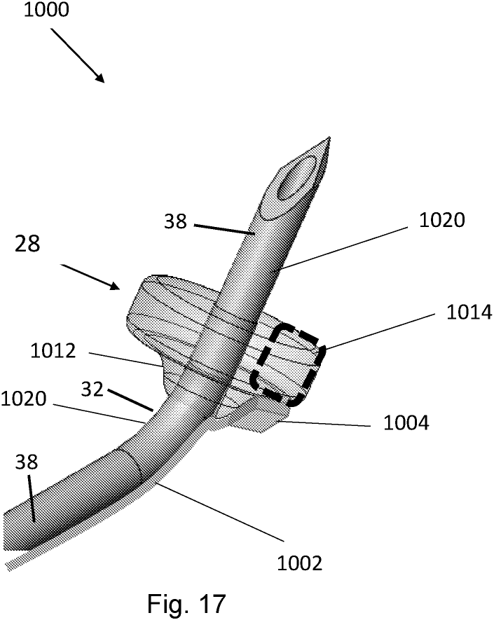

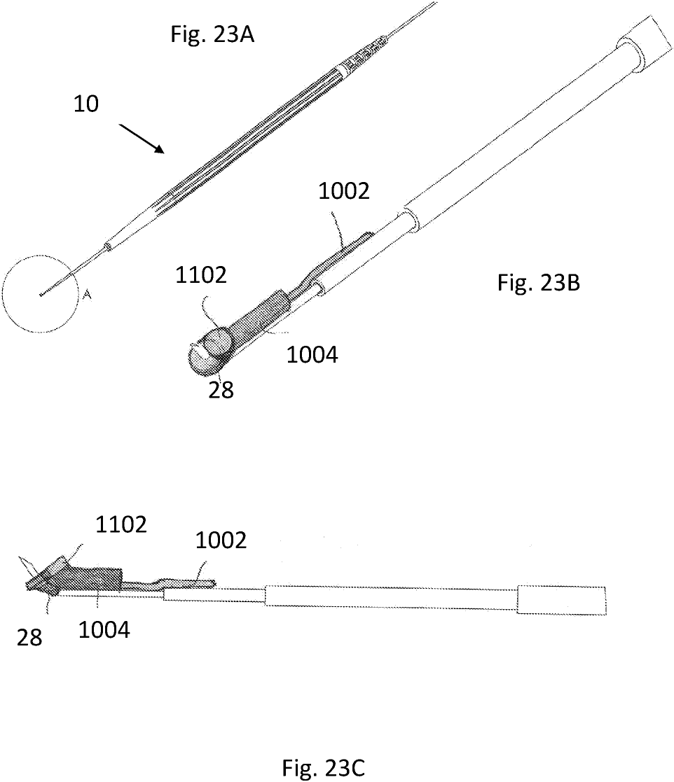

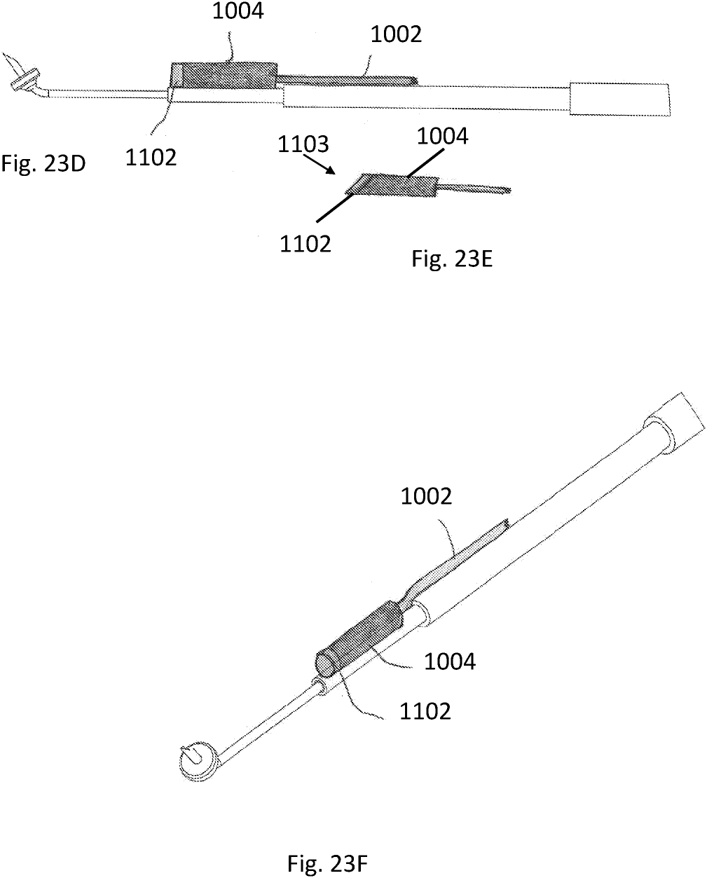

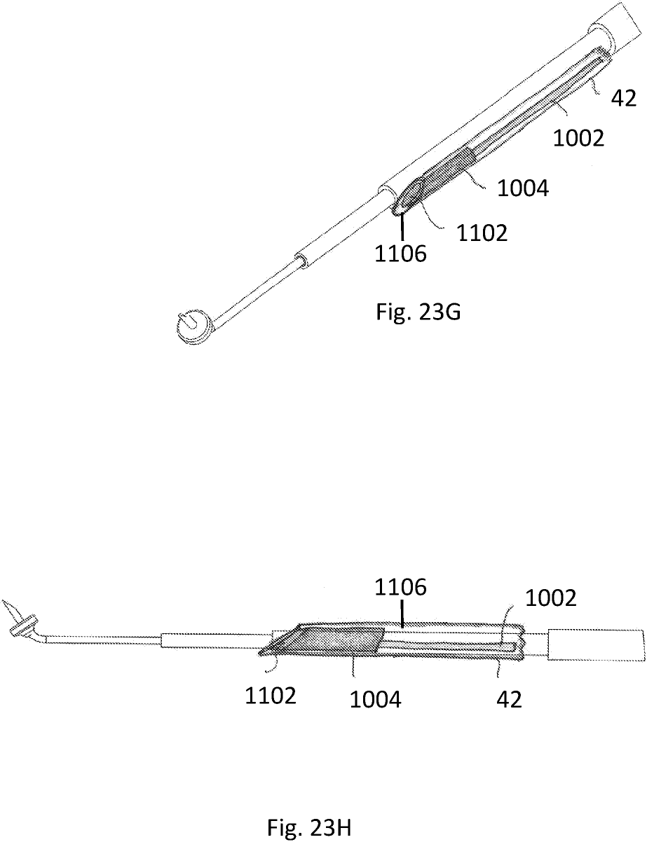

FIG. 17 illustrates a perspective view of a distal tip camera 1004 disposed behind the stopper 28 within a system 1000, according to aspects of the present embodiments. The stopper 28 may be attached to the outer surface 1020 of the bent portion 32 of needle 38. The distal tip camera 1004 may be operatively coupled with a wire 1002. In some embodiments, the distal tip camera 1004 may be disposed at a back surface 1012 of the stopper 28 (as shown in FIG. 17), while in some embodiments, the distal tip camera 1004 may be disposed behind the back surface 1012 of the stopper 28 (as shown in FIGS. 23D and 23F).

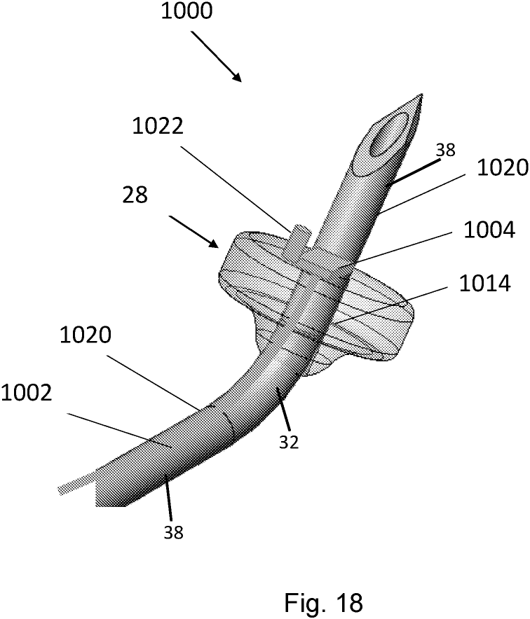

FIG. 18 illustrates a perspective view of a distal tip camera 1004 disposed within a system 1000, according to aspects of the present embodiments. The stopper 28 may be attached to the outer surface 1020 of the bent portion 32 of needle 38. The distal tip camera 1004 may be operatively coupled with a wire 1002. The distal tip camera 1004 may include or be operatively coupled to a light source 1022 (for example, an LED light source). In some embodiments, the distal tip camera 1004 and/or the light source 1022 may be disposed above the stopper 28. In some embodiments, the distal tip camera 1004 and/or the light source 1022 may be diposed at or embeded in the stopper 28 (not shown). In some embodiments, the distal tip camera 1004 and/or the light souce 1022 may be behind the stopper 28 (not shown).

Referring to FIGS. 15-18, the distal tip camera 1004 may be operatively coupled to the outside surface 1020 of the bent portion 32 of needle 38. In one or more embodiments, the stopper 28 may be transparent and/or include a transparent portion 1014 for the distal tip camera 1004 to see through the stopper 28 for monitoring and/or visualizing the target (for example, outer, middle, and/or inner ear). The transparent portion 1014 may include at least a transparent material (for example, plastic, thermoplastic, polymers, or and/or other suitable materials). In some embodiments, the transparent portion 1014 may be a part of the cavity 1010. In one or more embodiments, about 30% (or from about 20% to about 40%) of the stopper 28 may be removed such that the distal tip camera 1004 may be integrated and/or included in the needle sub-assembly 26 (FIG. 2 and FIG. 13).

Referring still to FIGS. 15-18, the distal tip camera 1004 may be operatively coupled via the wire 1002 to at least one of power supply for supplying power and/or communications to the distal tip camera 1004. In some embodiments, sections of the wire 1002 may be operatively attached to the outer surface 1020 of the needle 38. In some embodiments, sections of the wire 1002 may be operatively embedded within a portion of the device 10. For example, in one or more embodiments, the wire 1002 may be disposed outside of the bent needle 38 and telescoping hypotube needle support 24 while also extending at least partially internal to handle 12 of the device 10. In some embodiments, the wire 1002 may have a diameter of up to 10 mm. In some embodiments, the wire 1002 may have a diameter of up to 5 mm. In some embodiments, the wire 1002 may have a diameter of up to 3 mm. In some embodiments, the wire 1002 may have a diameter of up to 1 mm. In some embodiments, the wire 1002 may have a diameter of up to 0.8 mm. In some embodiments, the wire 1002 may have a diameter of up to 0.6 mm. In some embodiments, the wire 1002 may have a diameter of up to 0.4 mm. In some embodiments, the wire 1002 may have a diameter of up to 0.2 mm. In some embodiments, the wire 1002 may have a diameter of up to 0.1 mm. In some embodiments, the wire 1002 may have a diameter of up to 0.05 mm.

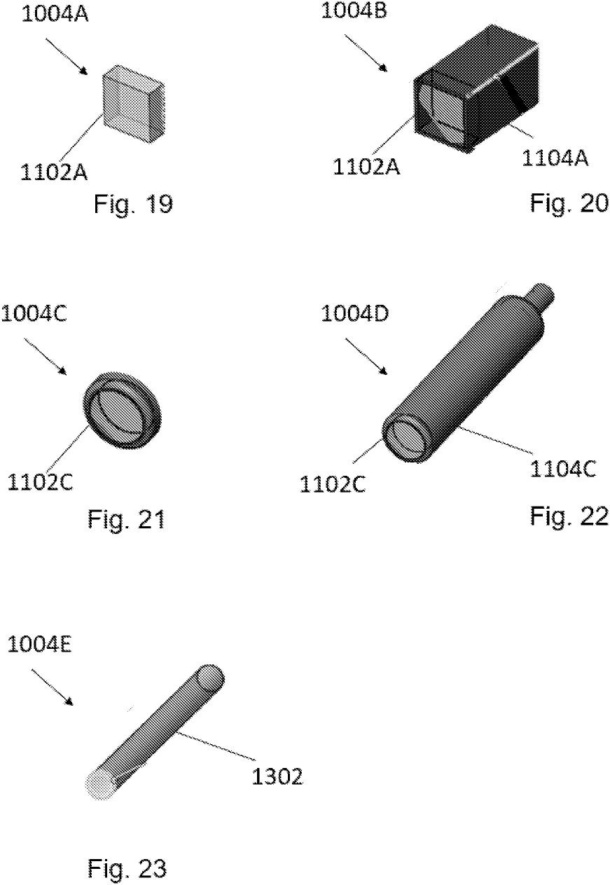

FIG. 19 illustrates a side view of a distal tip camera 1004A, according to aspects of the present embodiments. The distal tip camera 1004A may include an image sensor 1102A to receive, convey, and/or convert a signal (for example, an analog or digital signal) from the target (for example, the outer, middle, and/or inner ear) into an image. The image sensor 1102A may include a cuboid shape.

FIG. 20 illustrates a side view of a distal tip camera 1004B, according to aspects of the present embodiments. The distal tip camera 1004B may include a distal tip camera module 1104A that includes an image sensor 1102A, a processor (for example, a video processor, or a processor embedded in the distal tip camera 1004), and/or other elements (for example, driver and/or software packages) for the distal tip camera 1004B to access, operate, and/or process images (for example, images from the image sensor 1102A). In some embodiments, the distal tip camera module 1104A may include a package dimension of up to 0.7 mm × 0.7 mm with a z-height of up to 1.2 mm. In some embodiments, the distal tip camera module 1104A may include a package dimension of up to 1.1 mm × 1.1 mm with a z-height of up to 2.4 mm. In some embodiments, the distal tip camera module 1104A may include a package dimension of up to 1.5 mm × 1.5 mm with a z-height of up to 3 mm. In some embodiments, the distal tip camera module 1104A may include a package dimension of up to 2 mm × 2 mm with a z-height of up to 5 mm.

Referring to FIGS. 19-20, the image sensor 1102A may be up to 10 mm × 10 mm, 5 mm × 5 mm, 2 mm × 2 mm, 1.8 mm × 1.8 mm, 1.6 mm × 1.6 mm, 1.4 mm × 1.4 mm, 1.2 mm × 1.2 mm, 1 mm × 1 mm, 0.8 mm × 0.8 mm, 0.6 mm × 0.6 mm, 0.4 mm × 0.4 mm, 0.2 mm × 0.2 mm, 0.1 mm × 0.1 mm, or 0.05 mm × 0.05 mm with a height of up to 100, 20 10, 5, 3, 2, 1, 0.8, 0.6, 0.4, 0.2, or 0.1 mm.

Referring still to FIGS. 19-20, the image sensor 1102A may include an image array capable of capturing at least 10 × 10, 50 × 50, 100 × 100, 200 × 200, 400 × 400, 500 × 500 or 1000 × 1000 pixels resolution video at a frame rate of at least 5, 10, 20, 30, 50, 100, 500, or 1000 frames per second (fps).

Referring still to FIGS. 19-20, the image sensor 1102A may include an image area of at most 10 mm × 10 mm, 5 mm × 5 mm, 2 mm × 2 mm, 1.8 mm × 1.8 mm, 1.6 mm × 1.6 mm, 1.4 mm × 1.4 mm, 1.2 mm × 1.2 mm, 1 mm × 1 mm, 0.8 mm × 0.8 mm, 0.6 mm × 0.6 mm, 0.4 mm × 0.4 mm, 0.2 mm × 0.2 mm, 0.1 mm × 0.1 mm, or 0.05 mm × 0.05 mm. The image sensor 1102A may include low-light sensitivity of up to 10, 100, 500, 800, 1000, 1200, 1500, 2000, 3000, or 10,000 mV/lux-sec.

Referring still to FIGS. 19-20, the image sensor 1102A may include an optical format of up to 10, 5, 2, 1.8, 1.6, 1.4, 1.2, 1, 0.8, 0.6, 0.4, 0.2, 0.1, or 0.05 mm, and a pixel size of up to 10, 8, 6, 4, 3, 2.5, 2.2, 2, 1.8, 1.6, 1.4, 1.2, or 1 µm.

FIG. 21 illustrates a side view of a distal tip camera 1004C, according to aspects of the present embodiments. The distal tip camera 1004C may include an image sensor 1102C to receive, convey, and/or convert a signal (for example, an analog or digital signal) from the target (for example, outer, middle, and/or inner ear) into an image. The image sensor 1102C of FIG. 21 may include a shape of a chip or cylinder.

FIG. 22 illustrates a side view of a distal tip camera 1004D, according to aspects of the present embodiments. The distal tip camera 1004D may include a distal tip camera module 1104C that includes an image sensor 1102C, a processor (for example, a video processor, or an in-distal tip camera processor), and/or other elements (for example, driver and/or software packages) for the distal tip camera 1004D to access, operate, and/or process images (for example, images from the image sensor 1102C).

Referring to FIGS. 21-22, the image sensor 1102C may include an outer diameter of up to 10 mm, 5 mm, 2 mm, 1.8 mm, 1.6 mm, 1.4 mm, 1.2 mm, 1 mm, 0.8 mm, 0.6 mm, 0.4 mm, 0.2 mm, 0.1 mm, or 0.05 mm with a length of up to 100, 20, 10, 5, 3, 2, 1, 0.8, 0.6, 0.4, 0.2, or 0.1 mm.

Referring still to FIGS. 21-22, the image sensor 1102C may include an image array capable of capturing at least 10 × 10, 50 × 50, 100 × 100, 200 × 200, 400 × 400, 500 × 500 or 1000 × 1000 pixels resolution video at a frame rate of at least 5, 10, 20, 30, 50, 100, 500, or 1000 frames per second (fps).

Referring still to FIGS. 21-22, the image sensor 1102A may include an image area of at most 10 mm × 10 mm, 5 mm × 5 mm, 2 mm × 2 mm, 1.8 mm × 1.8 mm, 1.6 mm × 1.6 mm, 1.4 mm × 1.4 mm, 1.2 mm × 1.2 mm, 1 mm × 1 mm, 0.8 mm × 0.8 mm, 0.6 mm × 0.6 mm, 0.5 mm × 0.5 mm, 0.4 mm × 0.4 mm, 0.2 mm × 0.2 mm, 0.1 mm × 0.1 mm, or 0.05 mm × 0.05 mm. The image sensor 1102A may include a low-light sensitivity of up to 10, 100, 500, 800, 1000, 1200, 1500, 2000, 3000, or 10,000 mV/lux-sec.

Referring to FIGS. 19-22, the image sensors 1102A, C may be assembled using integrated circuits packages (for example, chip-scale packages (CSP)) that have an area of up to 0.1 mm × 0.1 mm, 0.3 mm × 0.3 mm, 0.6 mm × 0.6 mm, 0.9 mm × 0.9 mm, 1.2 mm × 1.2 mm, or 2 mm × 2 mm.

Referring still to FIGS. 19-22, the distal tip cameras 1004A-D may include a shutter (for example, a rolling shutter) (not shown). The distal tip cameras 1004A-D may operate at a temperature of -20° C. to 70° C. The distal tip cameras 1004A-D may include a field of view of at least 90, 100, 120, 130, or 150 degrees. In some embodiments, the distal tip cameras 1004A-D may include at least one internal lighting source, while in some embodiments, the distal tip cameras 1004A-D may be coupled externally to the at least one lighting source (for example, an LED lighting source 1022).

FIG. 23 illustrates a side view of an optical fiber 1302, according to aspects of the present embodiments. In the present disclosure, the optic fiber 1302 may be used as a light source, an image sensor 1004E, or both. The optical fiber 1302 may include at least a single optical fiber. In some embodiments, the optical fiber 1302 may have a diameter up to 5 mm and a length up to 10 m. In some embodiments, the optical fiber 1302 may have a diameter up to 1 mm and a length up to 10 m. In some embodiments, the optical fiber 1302 may have a diameter up to 0.5 mm and a length up to 10 m. In some embodiments, the optical fiber 1302 may have a diameter up to 0.4 mm and a length up to 10 m. In some embodiments, the optical fiber 1302 may have a diameter up to 0.3 mm and a length up to 1 m. In some embodiments, the optical fiber 1302 may have a diameter up to 0.2 mm and a length up to 1 m. In some embodiments, the optical fiber 1014 may have a diameter up to 0.1 mm and a length up to 0.1 m.

Referring to FIGS. 19-23, the distal tip camera 1004A-E and/or optical fiber 1302 may include or work with a processor (for example, a video processor) (not shown) with a dimension of up to 0.01 mm × 0.01 mm × 0.01 mm, 0.1 mm × 0.1 mm × 0.1 mm, 0.2 mm × 0.1 mm × 0.2 mm, 0.3 mm × 0.1 mm × 0.4 mm, 0.3 mm × 0.3 mm × 0.4 mm, 0.4 mm × 0.4 mm × 0.4 mm, or 1 mm × 1 mm × 1 mm. In some embodiments, the image sensors 1102A, C may be not larger or not longer than the distal tip camera modules 1104A, C.

Referring still to FIGS. 15-23, the distal tip camera 1004 may include at least one of the distal tip cameras 1004A-D, optical fiber 1302, and/or a combination thereof. The image sensor 1102 may include at least one of the image sensors 1102A, C, the optical fiber 1302, and/or a combination thereof.

Referring still to FIGS. 15-23, in one or more embodiments, the distal tip camera 1004 may include biocompatible, lead-free, auto-focus, disposable, reusable, low-noise, low power consumption, low heat, and/or low noise features for convenience. The distal tip camera 1004 may produce color, grey images, and/or a combination thereof.

Referring still to FIGS. 15-23, in some embodiments, the distal tip camera 1004 may include a working distance of up to 100 mm. In some embodiments, the distal tip camera 1004 may include a working distance of up to 50 mm. In some embodiments, the distal tip camera 1004 may include a working distance of up to 20 mm. In some embodiments, the distal tip camera 1004 may include a working distance of up to 10 mm. In some embodiments, the distal tip camera 1004 may include a working distance of up to 5 mm.

FIGS. 23A, 23B, and 23C illustrate a device 10 including a distal tip camera 1004 integrated into the stopper 28. FIG. 23A illustrates the device 10 with the tip portion circled at A, indicating the area of detail for FIGS. 23B and 23C. FIG. 23B illustrates a perspective view of the tip portion while FIG. 23C illustrates a side view of the tip portion. In the embodiment of FIGS. 23A, 23B, and 23C, the device 10 may include a lens or sensor 1102 and camera unit (for example, a distal tip camera 1004) integrated into the stopper 28. In some embodiments, the stopper 28 may be molded around the distal tip camera 1004. The distal tip camera 1004 may also be attached to the stopper 28 via adhesive, epoxy, and/or other suitable mechanisms. Wire 1002 provides power and a communication link to the distal tip camera 1004. In some embodiments, the wire 102 may be sized such that it is run or routed between two of the telescoping hypotubes (for example, between two of members 42A, 42B, 42C, or 42D of FIG. 9).

FIGS. 23D, 23E, and 23F illustrate a device 10 including a distal tip camera 1004 axially separated from the stopper 28 (for example, behind the stopper 28). In the embodiment of FIGS. 23D, 23E, and 23F, the device 10 may include a distal tip camera 1004 and lens 1102 that are mounted to one of the telescoping hypotubes (for example, member 42A, 42B, 42C, or 42D of FIG. 9), and may be positioned and/or angled such that they capture a representative view at the needle tip. The distal tip camera 1004 may be attached to the hypotube via adhesive, epoxy, welding and/or other suitable mechanisms. FIG. 23E includes a distal tip camera 1004 and lens or sensor 1102 in an angled configuration 1103.

FIGS. 23G and 23H illustrate the device 10 including the distal tip camera 1004, lens 1102, and wire 1002 mounted alongside one of the telescopic hypotubes (for example, member 42A, 42B, 42C, or 42D of FIG. 9). The distal tip camera 1004 may be attached to the hypotube via adhesive, epoxy, welding and/or other suitable mechanisms. In the embodiment of FIGS. 23G and 23H (as well as FIGS. 23A-23F), the device 10 may include a camera module casing 1106 externally encasing the distal tip camera 1004, lens 1102, and wire 1002.

Referring to FIGS. 15-23 (include FIGS. 23A-23H), in some embodiments, the device (including the camera) includes an overall effective diameter of less than about 10 mm, or from about 1 mm to about 10 mm, or from about 1 mm to about 8 mm, or from about 1 mm to about 5 mm, or from about 1 mm to about 4 mm, or from about 2 mm to about 4 mm, or from about 1 mm, to about 3 mm, or from about 1.5 mm to about 3 mm, or from about 1 mm to about 2.5 mm, and/or less than about 3 mm.

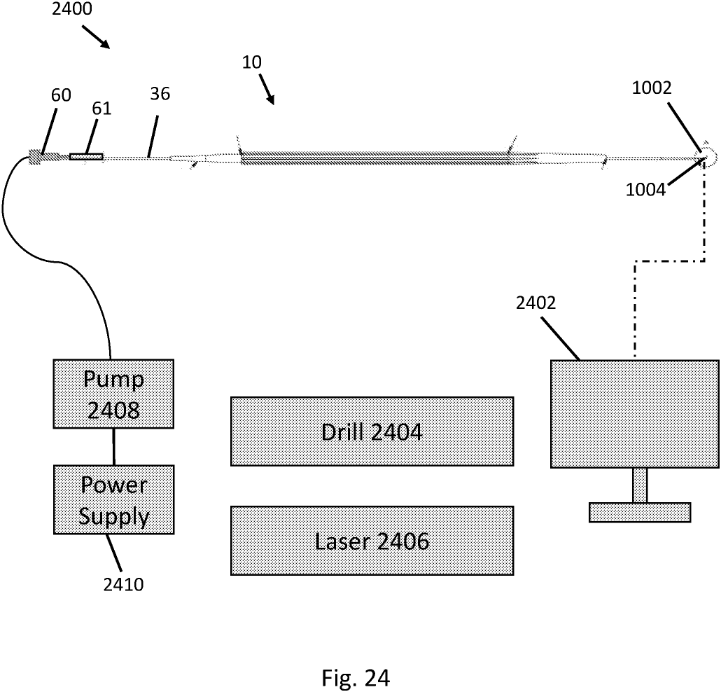

FIG. 24 illustrates a system 2400 including a device 10 according to aspects of the present disclosed embodiments. The system 2400 may also include a distal tip camera 1004, wire 1002, light source 1022, as described herein and/or monitor 2402. The monitor 2402 may be operatively coupled to the distal tip camera 1004. In some embodiments, the system 2400 may further include one or more syringes 60 for injecting fluids, one or more pumps 2408 fluidly connected to the syringe(s) or the tubing 36 (shown in FIGS. 3-5), a power supply 2410, sterilization equipment, sharps containers (for example, biohazard sharps container), drills 2404 (for example, otologic drill, and/or diamond drill), and/or lasers 2406 (for example, a KTP or CO₂ otologic laser) (not shown). In some embodiments, the system 2400 may include microscopes, endoscopes, and/or fiberscopes (not shown) (for example, if the system 2400 does not have a distal tip camera 1004). The syringe 60 may include a “Luer-Lok™ Syringe,” and may include a capacity from about 1 mL to about 100 mL, including various capacities and subranges therebetween including about 2 mL, about 2.5 mL, about 5 mL, about 10 mL, about 20 mL, about 30 mL, about 50 mL, and/or about 60 mL. The syringe 60 may include graduations of from about 0.002 mL to about 2.0 mL, or from about 0.005 mL to about 1.0 mL, or from about 0.01 mL to about 0.5 mL, or from about 0.05 mL to about 0.2 mL, or about 0.1 mL. The syringe 60 preferably and/or optionally meets ISO 13485, as well as other applicable safety and quality standards.

Still referring to FIG. 24, the pump 2408 is able to produce the desired flow and pressure conditions, in accordance with the present disclosure, and may be integrated with the syringe 60. The pump 2408 may include a Medfusion® 3500 Syringe Pump, and/or a Harvard Apparatus 70-2000, and/or may also include other designs, configurations, and arrangements (including pumps made from other OEMs), as long as the pump 2408 is able to produce the desired flow and pressure conditions, in accordance with the present disclosure. The pump 2408 may accommodate various syringe capacities including from about 1 mL to about 100 mL. The pump 2408 may also accommodate various flow rates including from about 1 mL/hr to about 50 mL/hr, or from about 2 mL/hr to about 40 mL/hr, or from about 3 mL/hr to about 25 mL/hr, or from about 4 mL/hr to about 20 mL/hr, or from about 5 mL/hr to about 15 mL/hr, or from about 8 mL/hr to about 12 mL/hr, or from about 0.5 mL/hr to about 15 mL/hr, or from about 1 mL/hr to about 12 mL/hr, or from about 2 mL/hr to about 10 mL/hr, or from about 2.5 mL/hr to about 8 mL/hr, or from about 3 mL/hr to about 7 mL/hr, or from about 4 mL/hr to about 6 mL/hr,. The pump 2408 may include a relative or gauge pressure operating range from about 0 psi to about 50 psi, and may be able to accommodate back-pressures in a range from about -300 mmHg to about +900 mmHg, while simultaneously delivering the desired flowrate to the device.

Referring still to FIG. 24, device 10 may be coupled to syringe 60 via a Luer lock 61 (for example, a “Luer-Lok™ Syringe,” which may be integrated with the syringe 60 and/or the pump 2408) to minimize air introduction during the delivery of fluid to the ear and to ensure appropriate connectivity with tubing 36 (for example, as shown in FIGS. 1, 3, 6, and/or 12). The Luer lock 61 (or Luer fitting) may include a custom bushing insert used to help de-air the system (i.e., used to help remove air from the system). The custom bushing may be molded silicone (or other suitable materials) and may be inserted on the inside of the Luer lock 61 (or fitting). The custom bushing may be generally cylindrical in shape with a center borehole (or lumen) through which the end of the PEEK tubing 36 may be disposed. The center borehole (or lumen) may include a conical entrance. The custom bushing helps to occupy “dead space” within the Luer lock 61 (or fitting) such that the amount of air in the interior of the Luer lock 61 (or fitting) is minimized. The Luer lock 61 may include Luer lock and/or Luer slip style connectors, and may include a slip tip. In addition, the Luer lock 61 may include male threads coupled to syringe 60 that interface with female threads coupled to needle 38, as well as other suitable leak-free connector and/or coupling configurations. Syringe 60 may be used for injecting fluids, e.g., a therapeutic fluid that contains a therapeutic agent that can be a small molecule or a biologic, e.g., an antibody or viral gene therapy. Syringe 60 may be packaged separately from other device components to accommodate device preparation. Syringe 60 may be sterilized. Device preparation may occur outside of the operating room (for example, in a pharmacy). Device preparation may occur inside of the operating room. Device preparation may be performed using a sterilization field and/or sterilization equipment. By including a distal tip camera 1004 on the device 10, the surgeon can see around corners, thereby avoiding the need to remove obstructions such as overhanging bone. In addition, using a delivery device 10 (or microcatheter) with an embedded distal tip camera 1004 allows the surgeon to operate with a single tool for piercing the round window, delivering a therapeutic fluid, and visualizing the outer, middle, and/or inner ear.

### Methods

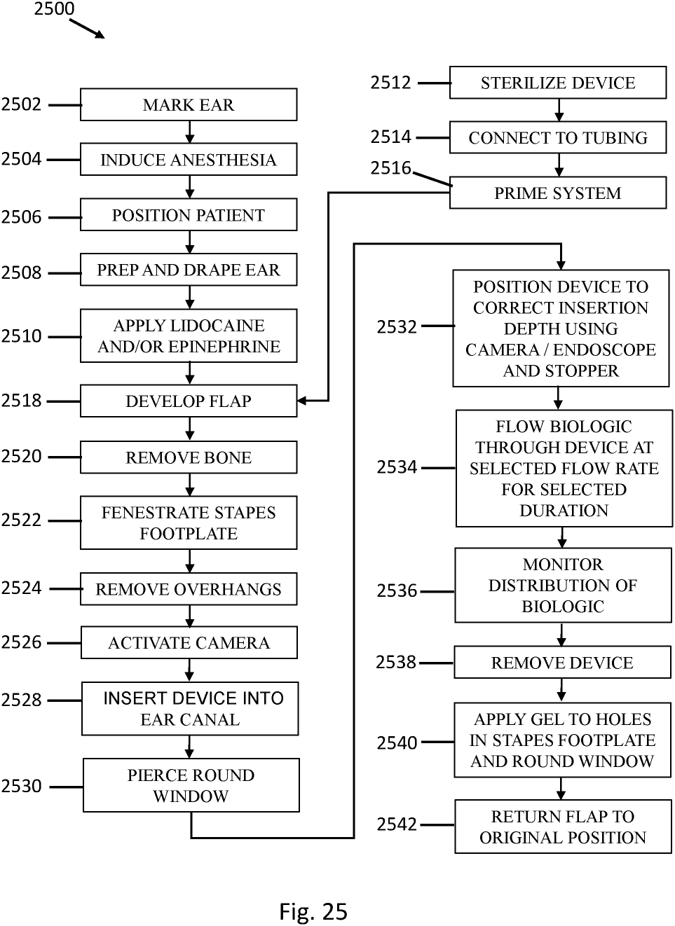

FIG. 25 illustrates a method 2500 that may be used to install the device 10 (or microcatheter) and deliver a fluid, e.g., a therapeutic fluid to the inner ear, according to aspects of the present embodiments. In some embodiments, the present disclosure describes a delivery approach that utilizes a minimally invasive, well-accepted surgical technique for accessing the middle ear and/or inner ear through the external auditory canal. The procedure includes opening one of the physical barriers between the middle and inner ear at the oval window, and subsequently using the delivery device 10 (or microcatheter) to deliver the therapeutic fluid (for example, including one or more biologics such as a viral gene therapy to treat a hearing disorder) at a controlled flow rate and in a fixed volume, via the round window membrane. FIG. 25 generally describes a surgical procedure as it applies to humans. However, similar methodologies and procedures also apply to mice, rodents, and non-human primates, as described in the following paragraphs.

The delivery device 10 may be placed in a sterile field of an operating room and the end of the tubing 36 may be removed from the sterile field and connected to the syringe 60 that has been loaded with the therapeutic fluid and mounted in the pump. After appropriate priming of the system 2400 in order to remove any air, the needle 38 may then be passed through the middle ear under visualization (surgical microscope, endoscope, and/or distal tip camera 1004). The needle 38 (or microneedle) may be used to puncture the RWM. The needle 38 may be inserted until the stopper 28 contacts the RWM. The device 10 may then be held in that position while the therapeutic fluid is delivered at a controlled flow rate to the inner ear. Once the delivery has been completed, the device 10 may be removed. The device 10 may be configured as a single-use disposable product. In other embodiments, the device 10 may be configured as a multi-use, sterilizable product, for example, with a replaceable and/or sterilizable needle sub-assembly 26. Single use devices 10 may be appropriately discarded (for example, in a biohazard sharps container) after administration is complete.

Referring to FIG. 25, the surgical procedure or method 2500 of delivering a therapeutic fluid to the inner ear of a patient may include, at step 2502, marking the ear to be treated with an indelible marker. At step 2504, the method 2500 may include inducing general anesthesia in the patient. At step 2506, the method 2500 may include positioning the patient in a supine position (that it, on his or her back) with the patient’s head turned to the side such that the marked ear is facing upward. At step 2508, the method 2500 may include prepping the ear with an antiseptic (such as povidone-iodine, iodopovidone, betadine, wokadine, pyodine, and/or other suitable antiseptics) and draping the ear and surrounding area (for example, covering the immediate area around the ear and/or otherwise creating a sterile barrier surrounding the ear while allowing access to the ear to minimize the risk of infection and/or contamination). At step 2510, the method 2500 may include applying lidocaine, epinephrine, and/or other anesthetics and adrenaline in a four-quadrant block to the ear canal. An operating microscope, an endoscope, and/or a distal tip camera 1004 may be used to apply the lidocaine and epinephrine precisely. Without limitation, the application of lidocaine and epinephrine may include a composition with about 1% lidocaine and epinephrine at a dilution of one part per eight thousand (1:8,000). In some embodiments, endoscopic surgeons may use higher or lower concentrations of lidocaine or epinephrine depending on the volume of the composition and the desired total dose of lidocaine or epinephrine per injection. In some embodiments the volume of the composition is less than 1 mL per injection.

Still referring to FIG. 25, steps 2512, 2514, and 2516 describe steps for prepping the system 2400. The system prepping steps (2512, 2514, and 2516) may occur at the same time as, before, and/or after the patient prepping steps (that is steps 2502, 2504, 2506, 2508, and 2510). At step 2512, the method 2500 may include sterilizing the device 10, for example via a sterilizing field (or, for example, via gamma irradiation or steam sterilization prior to packaging the device). At step 2514, the method 2500 may include connecting tubing 36 to the proximal end 16 of the device 10. At step 2516, the method 2500 may include priming and purging the system 2400 to ensure that air bubbles have been removed from all lines and to ensure that fluid suction is established within the pump. The system 2400 may be primed and purged using therapeutic fluid as the purging fluid. In one embodiment, a first amount of therapeutic fluid (for example, from about 8 µLto about 24 µL, or from about 12 µL to about 20 µL, or about 16 µL) is pushed through the device 10 until drops emerge from distal end 20 of the device. A second amount of therapeutic fluid (for example, about 3 µL to about 7 µL or about 5 µL) are then pushed through the device 10 to ensure the device is sufficiently purged. At step 2518, once the system and the patient have both been prepped, the method 2500 may include developing a posterior tympanomeatal flap such that the device 10 may reach the oval and round windows in the middle ear. At step 2520, the method 2500 may include removing a finite amount of bone at the junction of the bony canal and the tympanic membrane using a micro curette or drill. At step 2522, the method 2500 may include forming a hole or in the stapes footplate (or fenestrating the stapes footplate), which is located on the opposite side of the cochlea as the round window, thereby allowing for proper venting during solution delivery to the inner ear. The hole in the stapes footplate may be formed using an otologic laser (for example, a KTP or CO₂ otologic laser). At step 2524, the method 2500 may include removing overhanging bone (for example, a pseudomembrane or ledge of overhanging bone) as needed to expose the round window. Overhanging bone may be removed using a 1 mm diamond drill. At step 2526, the method 2500 may include activating a distal tip camera 1004 (which may be embedded in the device 10) to aid in the insertion and movement of the device 10 into and along the external auditory canal.

Referring still to FIG. 25, at step 2528, the method 2500 may include inserting the device 10 into the external auditory canal. At step 2528, the method 2500 may include the distal end 20 of the device 10 piercing the round window (step 2530) and passing through the round window to a depth of no more than about 1 mm (for example, to an insertion depth from about 0.7 mm to about 1 mm, or from about 0.8 mm to about 0.95 mm, or from about 0.85 mm to about 1.0 mm, or from about 0.85 mm to about 0.95 mm). The stopper 28 may be concentrically positioned about the bent portion 32 of the needle 38 (or microneedle) at an appropriate location to ensure the correct insertion depth of the needle 38 into the round window. In some embodiments, the distal tip camera 1004 (or endoscope and/or operating microscope) may be used (for example, in connection with the monitor 2402 or display screen to which it is communicatively coupled) by the surgeon to ensure the device is inserted to the correct insertion depth (step 2532). As such, the stopper 28 may be used primarily to ensure an insertion depth of 1.0 mm is not exceeded while the distal tip camera 1004 may be used to precisely position the device 10 prior to (and during) insertion of the needle 38 (or microneedle) into the round window. In other embodiments, the insertion depth may be greater than 1.0 mm and may include, for example, a depth of about 1.1 mm, about 1.2 mm, about 1.3 mm, about 1.4 mm, about 1.5 mm, about 1.6 mm, about 1.7 mm, as well as other subranges therebetween. At steps 2528, 2530, and/or 2532, the method 2500 may include adjusting the angle or orientation of the device 10 on the fly, as needed during the procedure. As such, even though the angle of the tip 34 of the device 10 is fixed relative to the handle portion 12, the orientation of the tip 34 relative to the RWM may be adjusted on the fly based on the angle or range of angles at which the surgeon orients the device 10. At step 2534, the method 2500 may include flowing the therapeutic fluid through the device 10 at a selected flow rate for a selected duration of time.

Still referring to FIG. 25, in some embodiments, the flow rate (or infusion rate) may include a rate of about 30 µL/min, or from about 25 µL/min to about 35 µL/min, or from about 20 µL/min to about 40 µL/min, or from about 20 µL/min to about 70 µL/min, or from about 20 µL/min to about 90 µL/min, or from about 20 µL/min to about 100 µL/min,. In some embodiments, the selected duration of time (that is, the time during which the therapeutic fluid is flowing) may be about 3 minutes, or from about 2.5 minutes to about 3.5 minutes, or from about 2 minutes to about 4 minutes, or from about 1.5 minutes to about 4.5 minutes, or from about 1 minute to about 5 minutes. In some embodiments, the total volume of therapeutic fluid that flows to the inner ear may be about 0.09 mL, or from about 0.08 mL to about 0.10 mL, or from about 0.07 mL to about 0.11 mL. In some embodiments, the treatment duration may be less than a minute (for example, from about 25 second to about 59 seconds, or from about 30 seconds to about 55 seconds, or from about 31 seconds to about 45 seconds). In some embodiments, the total volume of therapeutic fluid equates to from about 40% to about 50% of the volume of the inner ear. At step 2536, the method 2500 may include monitoring the distribution of the therapeutic fluid (or therapeutic agent) within the inner ear including the base, middle, and apex of the cochlea (for example, via the distal tip camera 1004, the endoscope and/or the operating microscope) to determine if the insertion depth of the device 10 within the round window should be adjusted (for example, deeper or shallower). For example, in one or more embodiments, the cumulative volume could be monitored while in other embodiments, fluorescent agents may be added to the therapeutic fluid which may then be excited and/or activated via optical fiber or distal tip camera 1004 such that the distribution of the therapeutic fluid to the inner ear may be visualized. At step 2538, the method 2500 may include removing the device 10. At step 2540, the method 2500 may include applying a skin treatment (for example, Healon (sodium hyaluronate) or hyaluronic acid) to both (or either of) the round window membrane and the stapes footplate to create functional seals of both areas (or either area) while healing occurs over a subsequent period, e.g., from about 24 hours to about 48 hours. At step 2542, the method 2500 may include returning the posterior tympanomeatal flap back to its original (biological) position. In some embodiments according to the present disclosure, method 2500 may include performing one or more steps in a different order than what is illustrated in FIG. 25 as well as additional steps not illustrated in FIG. 25. In some embodiments, one or more steps may be omitted and/or performed concurrently with at least one other step.

### CERTAIN DEFINITIONS

In order for the present disclosure to be more readily understood, certain terms are first defined below. Additional definitions for the following terms and other terms are set forth throughout the specification.

A device, composition, or method described herein as “comprising” one or more named elements or steps is open-ended, meaning that the named elements or steps are essential, but other elements or steps may be added within the scope of the composition or method. To avoid prolixity, it is also understood that any device, composition, or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any device, composition, or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

As used herein, “a” or “an” with reference to a claim feature means “one or more,” or “at least one.”

As used herein, “biocompatible” refers to materials that do not cause significant harm to living tissue when placed in contact with such tissue, e.g., in vivo. In some embodiments, materials are “biocompatible” if they are amenable to administration to the inner ear. In some embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects. In some embodiments, the materials used for the described devices and systems are biocompatible and tested to meet Class II biocompatibility requirement (e.g., devices with short term dwell times (less than 24 hours) and an indirect blood path).

As used herein, “disease”, “disorder”, and/or “condition” refers to any disease, disorder, and/or condition that may be treated by accessing the inner ear. In some embodiments, the disease, disorder, and/or condition is a hearing disorder (such as hearing loss). In some embodiments, the disease, disorder, and/or condition is a balance disorder. In some embodiments, the disease, disorder, and/or condition is a tumor such as an inner ear tumor. In some embodiments, the disease, disorder, and/or condition is a tumor such as a vestibular schwannoma. Other diseases, disorders, and/or conditions include, but are not limited to, acoustic neuromas, age-related dizziness and imbalance, autoimmune inner ear disease, benign paroxysmal positional vertigo, bilateral vestibular hypofunction, CANVAS syndrome, and chloesteatoma.

As used herein, “therapeutic fluid”, refers to a fluid composition that includes a therapeutic agent or a delivery modality for providing a therapeutic agent to the inner ear, e.g., a nucleic acid vector that encodes a therapeutic agent. A therapeutic agent can be any modality such as a small molecule or biologic that has the function of treating diseases or disorders, e.g., hearing diseases, disorders, and/or conditions, tumors, etc. In some embodiments, the therapeutic agent is a viral gene therapy. In some embodiments, the therapeutic agent is a therapeutic antibody. In some embodiments, the therapeutic agent is a therapeutic antisense oligonucleotide. In some embodiments, the therapeutic agent is a therapeutic nucleic acid (such as a RNA or DNA). In some embodiments, the therapeutic agent is a therapeutic miRNA. In some embodiments, the therapeutic agent is a therapeutic shRNA. In some embodiments, the therapeutic agent is a therapeutic CRISPR/Cas system that includes a Cas protein and guide molecule, e.g., a guide RNA. In some embodiments the therapeutic agent is delivered to the inner ear within the therapeutic fluid. In some embodiments the therapeutic agent is encoded by a delivery modality, e.g., a nucleic acid vector that is delivered to the inner ear within the therapeutic fluid. In some embodiments, the therapeutic agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a therapeutic fluid may be specially adapted for administration via injection, that is, e.g., an aqueous or non-aqueous solution or suspension.

As used herein, the term “pharmaceutically acceptable” which, for example, may be used in reference to a carrier used to formulate a therapeutic fluid as disclosed herein, means that a carrier is compatible with other ingredients of a fluid composition and not deleterious to a recipient thereof.

As used herein, the term “treat” (also “treatment” or “treating”) refers to any administration of a therapeutic agent (also “therapy”) that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a patient who does not exhibit signs of the relevant disease, disorder and/or condition and/or of a patient who exhibits only early signs of the disease, disorder, and/or condition. Alternatively, or additionally, such treatment may be of a patient who exhibits one or more established signs of the relevant disease, disorder and/or condition. In some embodiments, treatment may be of a patient who has been diagnosed as suffering from the relevant disease, disorder, and/or condition. In some embodiments, treatment may be of a patient known to have one or more susceptibility factors that are statistically correlated with increased risk of development of a given disease, disorder, and/or condition. In some embodiments the patient may be a human.

As used herein, the term “substantially” refers to the qualitative condition of exhibiting total or near-total extent or degree of a characteristic or property of interest.

### EQUIVALENTS

It is to be understood that while the disclosure has been described in conjunction with the detailed description thereof, the foregoing description is intended to illustrate and not limit the scope of the claims. Other aspects, advantages, and modifications are within the scope of the claims.

This written description uses examples to disclose the invention, including the best mode, and also to enable any person skilled in the art to practice the present embodiments, including making and using any devices or systems and performing any incorporated methods. The patentable scope of the present embodiments is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1-108. (canceled)

109. A device to deliver fluid to an ear, the device comprising:
a handle portion comprising a proximal end and a distal end;
a telescoping support coupled to the distal end of the handle portion;
a needle sub-assembly coupled to the distal end of the handle portion and coupled to the distal end of the telescoping support, the needle sub-assembly comprising a bent needle, the bent needle comprising:
an angled tip for piercing at least one membrane; and
a bent portion; and
tubing coupled to the proximal end of the handle portion,
wherein the bent needle extends through a hollow interior of the handle portion and fluidly connects directly to the tubing.

110. The device of claim 109, wherein the telescoping support comprises multiple nested hypotubes, each of the nested hypotubes comprising a gauge from 10 to 30, and each of the nested hypotubes comprising stainless steel.

111. The device of claim 109, comprising:
a strain relief feature coupled to the proximal end of the handle portion,
wherein the strain relief feature prevents kinking and/or deformation of the tubing, and
wherein the strain relief feature comprises layered extrusions.

112. The device of claim 109, comprising a distal tip camera positioned within the needle sub-assembly.

113. The device of claim 109, wherein the bent portion of the bent needle has a length from 0.5 mm to 5 mm, and
wherein the angle of the angled tip is from 20 degrees to 70 degrees, and wherein the angled tip projects from the bent portion of the bent needle to form an outlet for dispensing fluid.

114. The device of claim 109, the device comprising a stopper coupled to the bent needle, wherein the stopper is shaped and sized for positioning within the inner ear and controlling a distance that the angled tip projects into a cochlea.

115. The device of claim 109, wherein the stopper comprises a cylindrical disk shape with a diameter from 0.2 mm to 1.2 mm and a height from 0.2 mm to 1.0 mm,
wherein the stopper is molded in place onto the bent needle at a distance of 0.2 mm to 1.2 mm from a distal end of the angled tip,
wherein the stopper prevents the bent needle from being inserted into at least one membrane beyond a desired amount, and
wherein the stopper is seated around a stopper anchoring groove disposed within the bent needle.

116. The device of claim 109, wherein the handle portion further comprises:
machined grooves for tactility and control; and
a tapered portion disposed at the distal end of the handle, the telescoping support coupling to the tapered portion wherein the handle tapers down to the distal end such that the proximal end of the telescoping support is coupled to first distal end,
wherein the telescoping support tapers from an outer diameter of 0.2 inches or less at a proximal end to an outer diameter of 0.01 inches or more at a distal end.

117. The device of claim 109, wherein the tubing is coupled to the bent needle via a compression fit,
wherein the tubing comprises polyether ether ketone (PEEK),
wherein the tubing comprises an inner diameter from 0.0003 inches to 0.01 inches,
wherein the tubing comprises an outer diameter from 1/64 inches to 1/16 inches, and
wherein the tubing comprises a length greater than 20 inches.

118. The device of claim 111, further comprising:
at least one machined barb disposed at the proximal end of the handle portion, wherein the at least one machined barb interfaces with the strain relief feature and prevents axial movement between the handle portion and the strain relief feature; and
an annular brace disposed at an interface between the telescoping support and the handle portion.

119. A surgical procedure for delivering a therapeutic fluid to a portion of an inner ear of a patient comprising:
developing a posterior tympanomeatal flap;
creating an opening in a stapes footplate of the inner ear; and
piercing a round window with a needle positioned at the distal end of a fluid delivery device;
positioning the fluid delivery device at a desired insertion depth with the round window; and
flowing the therapeutic fluid through the fluid delivery devices to the inner ear.

120. The procedure of claim 119, further comprising:
activating at least one of a distal tip camera, an endoscope, and an operating microscope prior to piercing the round window; and
monitoring at least one of the flow rate of therapeutic fluid and the distribution of therapeutic fluid across the inner ear via at least one of the distal tip camera, the endoscope, and the operating microscope prior to piercing the round window,
wherein the distal tip camera comprises a communicative coupling to at least one monitor viewable by a surgeon during the procedure.

121. The procedure of claim 119, wherein developing a posterior tympanomeatal flap comprises cutting the posterior tympanomeatal using at least one of a micro curette and a drill.

122. The procedure of claim 119, further comprising:
prepping and draping the ear prior to developing the posterior tympanomeatal flap;
positioning the patient prior to prepping and draping the ear;
inducing anesthesia prior to positioning the patient;
marking the ear prior to inducing anesthesia;
connecting tubing between the fluid delivery device and an upstream pump prior to developing the posterior tympanomeatal flap;
sterilizing the fluid delivery device prior to developing the posterior tympanomeatal flap; and
priming the system prior to developing the posterior tympanomeatal flap.

123. The procedure of claim 119, wherein the therapeutic fluid comprises at least one viral gene therapy.

124. The procedure of claim 119, wherein the therapeutic fluid comprises adeno-associated virus (AAV) vector that comprises an Anc80 AAV vector or a coding region encoding hOTOF.

125. The procedure of claim 119, further comprising:
removing the fluid delivery device from the inner ear after flowing the therapeutic fluid through the fluid delivery device;
applying at least one skin treatment to at least one of the round window membrane and the stapes footplate after removing the fluid delivery device; and
returning the posterior tympanomeatal flap back to the original position after applying at least one skin treatment,
wherein the at least one skin treatment comprises at least one of sodium hyaluronate and hyaluronic acid.

126. The procedure of claim 119, further comprising removing bone from the junction of the bony canal and the tympanic membrane, and/or pseudomembrane overhanging bone, after developing the posterior tympanomeatal flap,
wherein removing bone comprises using at least one of a diamond drill and an otologic drill.

127. The procedure of claim 119, wherein creating an opening in the stapes footplate comprises creating an opening in the stapes footplate using a laser, wherein the laser comprises an otologic laser.

128. The procedure of claim 119, further comprising applying at least one of an anesthetic and an adrenaline to the ear canal of the patient prior to developing the posterior tympanomeatal flap.

129. The procedure of claim 120, wherein prepping the ear further comprises applying at least one antiseptic to the ear, wherein the at least one antiseptic comprises at least one of povidone-iodine, iodopovidone, betadine, wokadine, and pyodine.

130. A system for delivering a therapeutic fluid to a portion of an inner ear of a patient comprising:
means for performing a transcanal tympanotomy;
means for preparing a round window of the patient;
means for performing a laser-assisted micro-stapedotomy;
a delivery device configured to inject the therapeutic fluid to the inner ear;
a pump fluidly coupled to the delivery device to control flow of the therapeutic fluid through the delivery device;
means for applying sealant around at least one of the round window and an oval window of the patient; and
means for lowering a tympanomeatal flap of the patient to an anatomical position,
wherein the means for performing the laser-assisted micro-stapedotomy comprises at least one of a KTP otologic laser and a CO₂ otologic laser, and
wherein the therapeutic fluid comprises adeno-associated virus (AAV) vector that comprises an Anc80 AAV vector or a coding region encoding hOTOF.

131. The system of claim 130,
wherein the pump controls flow at a flow rate from 10 µL/min to 200 µL/min,
wherein the fluid delivery device is configured to deliver to the inner ear a total volume of therapeutic fluid in a range from 0.07 mL to 0.11 mL, and
wherein the fluid delivery device is configured to deliver the therapeutic fluid for a time duration in a range from 0.5 min to 5 min.
